SEQUENCE LISTING (49 sequences)

SEQ ID NO: 1 - protein, 6 aa, synthetic construct
  REGION 1..6: note = alpha chain CDR1
NSASQS

SEQ ID NO: 2 - protein, 6 aa, synthetic construct
  REGION 1..6: note = alpha chain CDR2
VYSSGN

SEQ ID NO: 3 - protein, 10 aa, synthetic construct
  REGION 1..10: note = alpha chain CDR3
AVISAGTALI

SEQ ID NO: 4 - protein, 109 aa, synthetic construct
  REGION 1..109: note = Amino acid sequence of alpha chain variable region
RKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSS

SEQ ID NO: 5 - DNA, 327 bp, synthetic construct
  misc_feature 1..327: note = Coding sequence of alpha chain variable region
  source 1..327: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaacgaagatggaaggtttacagcacagctcaatagagccagccagtatatttccctgctcatcagagactccaagctcagtgattcagccacctacctctgtgcggtgatctcggcaggaactgctctgatctttgggaagggaaccaccttatcagtgagttcc

SEQ ID NO: 6 - protein, 5 aa, synthetic construct
  REGION 1..5: note = beta chain CDR1
SGHDT

SEQ ID NO: 7 - protein, 6 aa, synthetic construct
  REGION 1..6: note = beta chain CDR2
YYEEEE

SEQ ID NO: 8 - protein, 15 aa, synthetic construct
  REGION 1..15: note = beta chain CDR3
ASSLGWRGGLYTEAF

SEQ ID NO: 9 - protein, 116 aa, synthetic construct
  REGION 1..116: note = Amino acid sequence ofbeta chain variable region
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVV

SEQ ID NO: 10 - DNA, 348 bp, synthetic construct
  misc_feature 1..348: note = Coding sequence of beta chain variable region
  source 1..348: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttggggtggcggggaggtctttacactgaagctttctttggacaaggcaccagactcacagttgta

SEQ ID NO: 11 - protein, 9 aa, synthetic construct
  REGION 1..9: note = Amino acid sequence of HPV16 E711-19 epitope
YMLDLQPET

SEQ ID NO: 12 - protein, 21 aa, synthetic construct
  REGION 1..21: note = leader sequence of the TCR alpha chain
MISLRVLLVILWLQLSWVWSQ

SEQ ID NO: 13 - protein, 19 aa, synthetic construct
  REGION 1..19: note = leader sequence of the TCR beta chain
MGPGLLCWALLCLLGAGLV

SEQ ID NO: 14 - protein, 141 aa, synthetic construct
  REGION 1..141: note = Amino acid sequence of WT human TRAC
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - protein, 141 aa, synthetic construct
  REGION 1..141: note = Amino acid sequence of human TRAC variant 1 (T48C)
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 141 aa, synthetic construct
  REGION 1..141: note = Amino acid sequence of human TRAC variant 2 (T48C + LVL mutation)
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 17 - protein, 137 aa, synthetic construct
  REGION 1..137: note = Amino acid sequence of WT murine TRAC
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 18 - protein, 137 aa, synthetic construct
  REGION 1..137: note = Amino acid sequence of murine TRAC variant 1 (T48C)
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 19 - protein, 137 aa, synthetic construct
  REGION 1..137: note = Amino acid sequence of murine TRAC variant 2 (T48C + LIVmutation)
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 20 - DNA, 411 bp, synthetic construct
  misc_feature 1..411: note = Coding sequence of murine TRAC variant 2 (T48C + LIV mutation)
  source 1..411: mol_type = other DNA
aatattcagaaccccgaaccagccgtatatcagttgaaggacccaagatctcaggatagtacactctgcctgttcaccgactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttca

SEQ ID NO: 21 - protein, 177 aa, synthetic construct
  REGION 1..177: note = Amino acid sequence of WT human TRBC1
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 22 - protein, 177 aa, synthetic construct
  REGION 1..177: note = Amino acid sequence of human TRBC1 variant (S57C)
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 23 - protein, 179 aa, synthetic construct
  REGION 1..179: note = Amino acid sequence of WT human TRBC2
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - protein, 179 aa, synthetic construct
  REGION 1..179: note = Amino acid sequence of human TRBC2 variant (S57C)
EDLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 25 - protein, 173 aa, synthetic construct
  REGION 1..173: note = Amino acid sequence of WT murine TRBC1
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 26 - protein, 173 aa, synthetic construct
  REGION 1..173: note = Amino acid sequence of murine TRBC1 variant (S57C)
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 27 - protein, 171 aa, synthetic construct
  REGION 1..171: note = Amino acid sequence of WT murine TRBC1'
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVRNR

SEQ ID NO: 28 - protein, 171 aa, synthetic construct
  REGION 1..171: note = Amino acid sequence of murine TRBC1' variant (S57C)
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVRNR

SEQ ID NO: 29 - protein, 173 aa, synthetic construct
  REGION 1..173: note = Amino acid sequence of WT murine TRBC2
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 30 - protein, 173 aa, synthetic construct
  REGION 1..173: note = Amino acid sequence of murine TRBC2 variant (S57C)
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 31 - DNA, 519 bp, synthetic construct
  misc_feature 1..519: note = Coding sequence of murine TRBC1 variant (S57C)
  source 1..519: mol_type = other DNA
gaggacctgcgcaacgtcaccccaccaaaggtcagtttgtttgagccatcaaaggcggagatcgccaacaaacagaaagctaccctcgtgtgcttggcccggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagc

SEQ ID NO: 32 - protein, 271 aa, synthetic construct
  REGION 1..271: note = Amino acid sequence of alpha chain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - protein, 271 aa, synthetic construct
  REGION 1..271: note = Amino acid sequence of alpha chain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSSNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 271 aa, synthetic construct
  REGION 1..271: note = Amino acid sequence of alpha chain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSSNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 35 - protein, 267 aa, synthetic construct
  REGION 1..267: note = Amino acid sequence of alpha chain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSSNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 36 - protein, 267 aa, synthetic construct
  REGION 1..267: note = Amino acid sequence of alpha chain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSSNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 37 - protein, 267 aa, synthetic construct
  REGION 1..267: note = Amino acid sequence of alpha chain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVISAGTALIFGKGTTLSVSSNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 38 - protein, 312 aa, synthetic construct
  REGION 1..312: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - protein, 312 aa, synthetic construct
  REGION 1..312: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 40 - protein, 314 aa, synthetic construct
  REGION 1..314: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 41 - protein, 314 aa, synthetic construct
  REGION 1..314: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 42 - protein, 308 aa, synthetic construct
  REGION 1..308: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 43 - protein, 308 aa, synthetic construct
  REGION 1..308: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 44 - protein, 306 aa, synthetic construct
  REGION 1..306: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVRNR

SEQ ID NO: 45 - protein, 306 aa, synthetic construct
  REGION 1..306: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVRNR

SEQ ID NO: 46 - protein, 308 aa, synthetic construct
  REGION 1..308: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 47 - protein, 308 aa, synthetic construct
  REGION 1..308: note = Amino acid sequence of beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGLYTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 48 - DNA, 63 bp, synthetic construct
  misc_feature 1..63: note = Nucleotide sequence encoding leader sequence of the TCR alpha chain
  source 1..63: mol_type = other DNA
atgatatccttgagagttttactggtgatcctgtggcttcagttaagctgggtttggagccaa

SEQ ID NO: 49 - DNA, 57 bp, synthetic construct
  misc_feature 1..57: note = Nucleotide sequence encoding leader sequence of the TCR beta chain
  source 1..57: mol_type = other DNA
atgggccccgggctcctctgctgggcactgctttgtctcctgggagcaggcttagtg

(19) United States
(12) Patent Application Publication
(10) Pub. No.: US 2024/0424101 A1
(43) Pub. Date: Dec. 26, 2024

(54) ANTIGEN BINDING PROTEINS AND USES THEREOF

(71) Applicant: CORREGENE BIOTECHNOLOGY CO., LTD., Beijing (CN)

(72) Inventors: Dong JIANG, Beijing (CN); Jianghua WANG, Beijing (CN); Xingwang XIE, Beijing (CN); Xueyan WANG, Beijing (CN); Jinglei BI, Beijing (CN); Yunqiang SHI, Beijing (CN); Jiahui ZHAI, Beijing (CN); Jinlong CONG, Beijing (CN)

(21) Appl. No.: 18/690,110

(22) PCT Filed: Sep. 7, 2022
(86) PCT No.: PCT/CN2022/117497

§ 371 Date: Mar. 7, 2024

(30) Foreign Application Priority Data
Sep. 7, 2021 (CN) 202111043194.X

(51) Int. Cl. A61K 39/00; A61P 35/00; C07K 14/725

(52) U.S. Cl. CPC A61K 39/464838; A61K 39/4611; A61K 39/4632; A61P 35/00; C07K 14/7051

(57) ABSTRACT

The present disclosure discloses antigen binding proteins, nucleic acids encoding the antigen binding proteins of the present disclosure, vectors including the nucleic acids of the present disclosure, cells including the antigen binding proteins, nucleic acids or vectors of the present disclosure, methods of preparing the cells of the present disclosure. The disclosure also discloses conjugates or compositions including the antigen binding proteins of the disclosure, and methods of preventing and/or treating diseases by using the antigen binding proteins or cells of the disclosure, and methods of detecting the presence of diseases in a subject.

### CROSS-REFERENCE TO RELATED APPLICATIONS

This application is an U.S. national phase application under 35 U.S.C. § 371 based upon international patent application No. PCT/CN2022/117497 filed on 7 Sep. 2022, which itself claims the benefit of Chinese Patent Application No.: 202111043194.X filed on Sep. 7, 2021. The entire contents of the above identified applications are incorporated by reference for all purposes.

SEQUENCE LISTING

This application contains a Sequence listing in computer readable form. The computer readable form is xml format and is incorporated herein by reference in its entirety. Said xml file, created on 26 Jul. 2022, is named C22W1989.xml and is 71,781 bytes in size.

### FIELD OF INVENTION

The present disclosure relates to antigen binding proteins, in particular T-cell receptors (TCRs), and uses thereof, in particular the use in the prevention, treatment or detection of HPV-positive diseases.

### BACKGROUND

Cervical cancer ranks fourth in the incidence of female tumors worldwide. The World Health Organization (WHO) estimates that there are 604,000 new cases and 342,000 deaths for cervical cancer worldwide. The incidence of cervical cancer in China has been on the rise in recent years, with about 109,000 new cases and 59,000 deaths for cervical cancer each year. The 5-year survival rate for metastatic, advanced cervical cancer is only about 10%. Almost all cervical cancers are associated with infection of high-risk human papilloma virus (HPV) types, of which HPV16 is the most common type, accounting for 60% of all high-risk HPV types. HPV infection can also cause head and neck cancer, oropharyngeal cancer, esophageal adenocarcinoma, anal cancer, vulvar cancer, penile cancer, among others. Currently, there is no effective treatment for HPV infection and the resulting malignancies. Prophylactic vaccines induce specific neutralizing antibodies generated by the organism against new viral infection, but cannot clear existing HPV infection. Although therapeutic vaccines, which are still in clinical trials, can slow down the progression of precancerous lesions to a certain extent, they are difficult to be therapeutically effective against advanced tumors.

TCR-T confers new antigen recognition specificity to T cells by cloning a TCR that specifically recognizes a specific human leukocyte antigen (HLA)-tumor antigen peptide complex through corresponding experimental techniques, followed by delivering a TCR gene coding sequence to these T cells by means of gene delivery such as lentivirus. Patient-derived T cells that are transduced with the TCR gene in vitro and amplified in large amount are able to effectively recognize tumor cell-specific antigens. These T cells are transfused back to the patient to specifically kill tumor cells and exert anti-tumor activity. Since both intracellular and extracellular antigens can be recognized by TCRs after HLA presentation, TCR-T can target most tumor-specific antigens, especially can recognize those intracellular tumor antigens (about 90% of all antigens), and thus TCR-T has the potential to be used in the treatment of almost all tumors, especially various solid tumors.

E6 and E7 proteins of HPV16 virus are important oncogenes that drive development and progression of cervical cancer, and almost all HPV16-positive tumor cells stably express these two antigens. HPV-associated cervical cancer can be effectively treated if E6 and E7 protein-positive tumor cells can be effectively recognized and killed. Previous studies have found that E6and E7 antigens of HPV16 virus can be effectively presented by HLA molecules and become potential antigenic targets recognized by specific TCRs. Therefore, there is a need to develop TCR products that specifically target HPV16, in particular, the HPV16 E7 antigen.

### SUMMARY OF THE INVENTION

The present disclosure provides novel antigen binding proteins that specifically bind to an HPV16 E7 antigen, in particular an HPV16 E7₁₁₋₁₉ epitope or the complex of the epitope with an major histocompatibility complex (MHC) molecule, such as the complex of HPV16 E7₁₁₋₁₉ epitope with HLA-A*02. The antigen binding proteins of the present disclosure can be in the form of a TCR or antigen-binding fragments thereof. The antigen binding proteins of the present disclosure are capable of binding a target antigen peptide with high affinity, have good expression stability, and are capable of mediating specific killing effect of effector cells against antigen-positive target cells.

Accordingly, in one aspect, the present disclosure provides an antigen binding protein comprising a T cell receptor (TCR) alpha chain variable region and a TCR beta chain variable region, wherein the TCR alpha chain variable region comprises a complementarity determining region 3 (CDR3) having the amino acid sequence: AVISAGTALI (SEQ ID NO:3), or a functional variant thereof formed by insertion, deletion or substitution of one or more amino acids, wherein the antigen binding protein binds to an epitope comprising the amino acid sequence of YMLDLQPET (SEQ ID NO: 11) or a complex of the epitope with an MHC molecule.

In another aspect, the present disclosure provides an antigen binding protein comprising a T cell receptor (TCR) alpha chain variable region and a TCR beta chain variable region, wherein the TCR beta chain variable region comprises a CDR3 having the amino acid sequence: ASSLGWRGGLYTEAF (SEQ ID NO:8), or a function variant thereof formed by insertion, deletion or substitution of one or more amino acids, wherein the antigen binding protein binds to an epitope comprising the amino acid sequence of YMLDLQPET (SEQ ID NO:11) or a complex of the epitope with an MHC molecule.

In yet another aspect, the present disclosure provides an antigen binding protein comprising a T cell receptor (TCR) alpha chain variable region and a TCR beta chain variable region, wherein the TCR alpha chain variable region comprises a CDR3 having the amino acid sequence: AVISAGTALI (SEQ ID NO:3), or a functional variant thereof formed by insertion, deletion or substitution of one or more amino acids, and the TCR beta chain variable region comprises a CDR3 having the amino acid sequence: ASSLGWRGGLYTEAF (SEQ ID NO:8), or a functional variant thereof formed by insertion, deletion or substitution of one or more amino acids, wherein the antigen binding protein binds to an epitope comprising the amino acid sequence of YMLDLQPET (SEQ ID NO: 11) or a complex of the epitope with an MHC molecule.

In some embodiments, the MHC molecule is the HLA-A*02 type, such as HLA-A*02:01type, HLA-A*02:03 type, HLA-A*02:05 type, HLA-A*02:06 type, HLA-A*02:07 type, HLA-A*02:10 type, or HLA-A*02:11 type.

In some embodiments, the TCR alpha chain variable region comprises a CDR3 having an amino acid sequence as shown in SEQ ID NO: 3, and/or the TCR beta chain variable region comprises a CDR3 having an amino acid sequence as shown in SEQ ID NO:8.

In some embodiments, the TCR alpha chain variable region comprises complementarity determining region 1 (CDR1), complementarity determining region 2 (CDR2), and CDR3 having the amino acid sequences as shown in SEQ ID NO:1, SEQ ID NO:2, and SEQ ID NO:3, respectively, or a functional variant thereof formed by insertion, deletion, or substitution of one or more amino acids; and/or the TCR beta chain variable region comprises beta chain CDR1,CDR2, and CDR3 having the amino acid sequences as shown in SEQ ID NO:6, SEQ ID NO:7 and SEQ ID NO:8, respectively, or a functional variant thereof formed by insertion, deletion or substitution of one or more amino acids.

In another aspect, the present disclosure provides an antigen binding protein comprising a T cell receptor (TCR) alpha chain variable region and a TCR beta chain variable region, wherein the TCR alpha chain variable region comprises CDR1, CDR2, and CDR3 having the amino acid sequences as shown in SEQ ID NO:1, SEQ ID NO:2, and SEQ ID NO:3, respectively; and/or the TCR beta chain variable region comprises beta chain CDR1, CDR2 and CDR3 having the amino acid sequences as shown in SEQ ID NO:6, SEQ ID NO: 7 and SEQ ID NO:8, respectively.

In some embodiments, the TCR alpha chain variable region comprises CDR1, CDR2, and CDR3 having the amino acid sequences as shown in SEQ ID NO:1, SEQ ID NO:2, and SEQ ID NO: 3, respectively; and the TCR beta chain variable region comprises beta chain CDR1, CDR2, and CDR3 having the amino acid sequences as shown in SEQ ID NO:6, SEQ ID NO:7, and SEQ ID NO: 8, respectively.

In some embodiments of the antigen binding protein of the present disclosure, the TCR alpha chain variable region comprises an amino acid sequence having at least 80%, at least 85%, at least 90%, at least 95%, or 100% sequence identity to SEQ ID NO:4, and/or the TCR beta chain variable region comprises an amino acid sequence having at least 80%, at least 85%, at least 90%, at least 95%, or 100% sequence identity to SEQ ID NO:9.

In some embodiments, the TCR alpha chain variable region comprises an amino acid sequence having at least 80%, at least 85%, at least 90%, at least 95%, or 100% sequence identity to SEQ ID NO:4, and the TCR beta chain variable region comprises an amino acid sequence having at least 80%, at least 85%, at least 90%, at least 95%, or 100% sequence identity to SEQ ID NO:9.

In some embodiments, the TCR alpha chain variable region comprises the amino acid sequence as shown in SEQ ID NO:4, and/or the TCR beta chain variable region comprises the amino acid sequence as shown in SEQ ID NO:9. In some embodiments, the TCR alpha chain variable region comprises the amino acid sequence as shown in SEQ ID NO:4, and the TCR beta chain variable region comprises the amino acid sequence as shown in SEQ ID NO:9.

In some embodiments, the TCR alpha chain variable region is contained on a first polypeptide and the TCR β chain variable region is contained on a different second polypeptide.

In some embodiments, the TCR alpha chain variable region and the TCR beta chain variable region are contained on a single polypeptide.

In some embodiments, the antigen binding protein is soluble or membrane-bound.

In some embodiments, the antigen binding protein is selected from a TCR, a chimeric antigen receptor (CAR), a Fc polypeptides, or an antigen-binding fragment thereof.

In some embodiments, the antigen binding protein is a TCR or an antigen-binding fragment thereof, and the antigen binding protein further comprises a TCR constant region or a fragment thereof.

In some embodiments, the TCR constant region is a murine constant region or a human constant region.

In some embodiments, the TCR constant region comprises a TCR alpha chain constant region and/or a TCR beta chain constant region; in an embodiment, the TCR alpha chain constant region and/or TCR beta chain constant region comprises at least one cysteine mutation relative to a wild-type sequence to form a disulfide bond between the TCR alpha chain and the TCR beta chain.

In some embodiments, the TCR alpha chain constant region comprises an amino acid sequence having at least 80%, at least 85%, at least 90%, at least 95%, or 100% sequence identity to any one of SEQ ID NOs: 14-19, and/or the TCR beta chain constant region comprises an amino acid sequence having at least 80%, at least 85%, at least 90%, at least 95%, or 100% sequence identity to any one of SEQ ID NOs: 21-30.

In some embodiments, the TCR alpha chain constant region comprises the amino acid sequence as shown in SEQ ID NO:19 and the TCR beta chain constant region comprises the amino acid sequence as shown in SEQ ID NO:26.

In some embodiments, the fragment of the TCR constant region is an extracellular segment of the TCR constant region.

In some embodiments, the antigen binding protein further comprises a transmembrane region and/or a cytoplasmic region.

In some embodiments, the antigen binding protein comprises a TCR alpha chain comprising an amino acid sequence having at least 80%, at least 85%, at least 90%, at least 95%, or 100% sequence identity to any one selected from SEQ ID NOs: 32-37; and/or a TCR beta chain comprising an amino acid sequence having at least 80%, at least 85%, at least 90%, at least 95%, or 100% sequence identity to any one selected from SEQ ID NOs: 38-47. In some embodiments, the antigen binding protein comprises a TCR alpha chain comprising an amino acid sequence having at least 80%, at least 85%, at least 90%, at least 95%, or 100% sequence identity to any one selected from SEQ ID NOs: 32-37; and a TCR beta chain comprising an amino acid sequence having at least 80%, at least 85%, at least 95%, or 100% sequence identity to any one selected from SEQ ID NOs: 38-47.

In some embodiments, the antigen binding protein further comprises an intracellular signaling region. In some embodiments, the antigen binding protein further comprises one or more antigen-binding region(s) that bind(s) to other antigens or epitopes.

In some embodiments, the antigen binding protein is isolated or purified.

In a further aspect, the present disclosure provides a nucleic acid encoding the antigen binding protein of the present disclosure.

In some embodiments, the nucleic acid comprises a nucleotide sequence having at least 80%, at least 85%, at least 90%, at least 95%, or 100% sequence identity to SEQ ID NO:5, and/or a nucleotide sequence having at least 80%, at least 85%, at least 90%, at least 95%, or 100% sequence identity to SEQ ID NO:10. In some embodiments, the nucleic acid comprises an amino acid sequence as shown in SEQ ID NO:5 and a nucleotide sequence as shown in SEQ ID NO: 10.

In some embodiments, the nucleic acid further comprises a nucleotide sequence having at least 80%, at least 85%, at least 90%, at least 95%, or 100% sequence identity to SEQ ID NO:20, and/or a nucleotide sequence having at least 80%, at least 85%, at least 90%, at least 95%, or 100% sequence identity to SEQ ID NO:31. In some embodiments, the nucleic acid further comprises a nucleotide sequence as shown in SEQ ID NO:20 and a nucleotide sequence as shown in SEQ ID NO:31.

In another aspect, the present disclosure provides a vector comprising the nucleic acid of the present disclosure.

In some embodiments, the vector is selected from the group consisting of lentiviral vectors, retroviral vectors, plasmids, DNA vectors, mRNA vectors, transposon-based vectors, and artificial chromosomes.

In yet another aspect, the present disclosure provides a cell comprising the antigen binding protein, the nucleic acid, or the vector according to the present disclosure.

In some embodiments, the cell is selected from the group consisting of lymphocytes (e.g., T cells, natural killer cells (NK cells)), monocytes (e.g., peripheral blood mononuclear cells (PBMCs)), and stem cells. In some embodiments, the stem cells are lymphoid progenitor cells or induced pluripotent stem cells (iPSCs).

In some embodiments, the cell is T cell. In some embodiments, the T cell does not express an endogenous TCR.

In another aspect, the present disclosure provides a method of preparing the cell of the present disclosure comprising transducing or transfecting the cell with the vector of the present disclosure.

In some embodiments, the method further comprises amplifying and/or activating the cells prior to or after the transduction or transfection.

In another aspect, the present disclosure provides a conjugate comprising the antigen binding protein of the present disclosure and an active agent coupled or conjugated to the antigen binding protein.

In some embodiments, the active agent is selected from the group consisting of a detectable marker, an immunostimulatory molecule and a therapeutic agent. In an embodiment, the detectable marker is selected from the group consisting of biotins, streptavidin, enzymes or catalytically active fragments thereof, radionuclides, nanoparticles, paramagnetic metal ions, nucleic acid probes, contrast agents, and fluorescent, phosphorescent or chemiluminescent molecules. In an embodiment, the immunostimulatory molecule is selected from the group consisting of cytokines, chemokines, platelet factors, and complement initiators. In an embodiment, the therapeutic agent is selected from the group consisting of immunomodulators, radioactive compounds, enzymes, chemotherapeutic agents, and toxins.

In another aspect, the present disclosure provides a composition comprising the antigen binding protein, the nucleic acid, the vector, or the cell of the present disclosure. In an embodiment the composition further comprises a pharmaceutically acceptable carrier or excipient.

In some embodiments, the composition further comprises a second therapeutic agent. In an embodiment the second therapeutic agent is selected from the group consisting of antibodies, chemotherapeutic agents and small molecule drugs.

In yet another aspect, the present disclosure provides a method of treating or preventing an HPV positive disease in a subject comprising administering to the subject an effective amount of the antigen binding protein of the present disclosure.

In another aspect, the present disclosure provides a method of treating or preventing an HPV positive condition in a subject comprising administering to the subject an effective amount of the cell of the present disclosure.

In some embodiments of the treatment method of the present disclosure, the cell is autologous or allogeneic for the subject.

In some embodiments, the method comprises (i) isolating a sample containing cells from the subject; (ii) transducing or transfecting the cells with the vector of the present disclosure; and (iii) administering the cells obtained in item (ii) to the subject. In some embodiments, the method further comprises knocking out an endogenous TCR in the cells after item (i) and prior to item (ii).

In some embodiments, the HPV positive disease is selected from the group consisting of an HPV infection, an HPV premalignancy and an HPV cancer. In an embodiment, the cancer is selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

In some embodiments, the method further comprises administering a second therapeutic agent. In an embodiment, the second therapeutic agent is selected from the group consisting of antibodies, chemotherapeutic agents and small molecule drugs.

In some embodiments of the treatment method of the present disclosure, the subject has an HLA-A*02 allele, such as an HLA-A*02:01, HLA-A*02:03, HLA-A*02:05, HLA-A*02:06, HLA-A*02:07, HLA-A*02:10, or HLA-A*02:11 allele.

In another aspect, the present disclosure provides a method of detecting (e.g., diagnosing) an HPV positive disease in a subject, wherein the method comprises (i) contacting a sample obtained from the subject with the antigen binding protein, the cell, or the conjugate of the present disclosure; and (ii) detecting the presence of an HPV antigen in the sample, wherein the presence of the HPV antigen is indicative of the HPV positive disease. In some embodiments, the HPV positive disease is selected from the group consisting of an HPV infection, an HPV premalignancy and an HPV cancer. In an embodiment, the cancer is selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

In yet another aspect, the present disclosure provides a kit comprising the antigen binding protein or the conjugate according to the present disclosure, the kit is used to detect the presence of a positive epitope in a sample to be tested, wherein the epitope is an epitope comprising YMLDLQPET (SEQ ID NO:11).

In one specific embodiment, the kit is used to detect (e.g., diagnose) an HPV-positive disease in a subject, and the kit comprises the antigen binding protein or the conjugate of the present disclosure. In some embodiments, the HPV-positive disease is selected from the group consisting of an HPV infection, an HPV premalignancy, and an HPV cancer. The cancer may be selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

In another aspect, the present disclosure provides use of the antigen binding protein, the nucleic acid, the vector, the cell, or the composition of the present disclosure in the preparation of a medicament for treating or preventing an HPV-positive disease in a subject.

In yet another aspect, the present disclosure provides the antigen binding protein, the nucleic acid, the vector, the cell, or the composition of the present disclosure for use in treating or preventing an HPV-positive disease in a subject.

In another aspect, the present disclosure provides use of the antigen binding protein or the conjugate of the present disclosure in the preparation of a kit for detecting (e.g., diagnosing) the presence of a positive epitope in a sample to be tested, wherein the epitope is an epitope comprising YMLDLQPET (SEQ ID NO:11), e.g., for detecting (e.g., diagnosing) an HPV-positive disease in a subject.

In yet another aspect, the present disclosure provides the antigen binding protein or the conjugate of the present disclosure for use in detecting (e.g., diagnosing) the presence of a positive epitope in a sample to be tested, wherein the epitope is an epitope comprising YMLDLQPET (SEQ ID NO:11), for example, for use in detecting (e.g., diagnosing) an HPV-positive disease in a subject.

In some embodiments of the uses of the present disclosure, the HPV-positive disease is selected from the group consisting of an HPV infection, an HPV premalignancy, and an HPV cancer. In an embodiment, the cancer is selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer. In some embodiments, the cancer is HPV16 positive. In some embodiments, the subject has an HLA-A*02:01 allele, such as an HLA-A*02:01, HLA-A*02:03, HLA-A*02:05, HLA-A*02:06, HLA-A*02:07, HLA-A*02:10, or HLA-A*02:11 allele.

### DESCRIPTION OF THE DRAWINGS

FIG. 1 shows the screening process and initial characterization of HPV16 E7-specific TCRs. (A) Screening and cloning of HPV16 E7-specific TCRs; (B) A HPV16 E7-specific TCR (CRTE7A2) specifically recognizes the HLA-A*02:01-presented HPV16 E7₁₁₋₁₉ epitope as determined by flow cytometry.

FIG. 2 shows the binding affinity of CRTE7A2 for the target antigen peptide (YMLDLQPET) (SEQ ID NO:11) as determined by flow cytometry. KITE-439 was used as a control.

FIG. 3 shows the phenotypes of CRTE7A2 and KITE-439 TCR-T cells as determined by flow cytometry.

FIG. 4 shows the specific killing of CRTE7A2 TCR-T cells against antigen-positive tumor cells. (A) The killing effect of CRTE7A2 TCR-T cells against positive target cells and negative target cells; (B) The specific killing effect of CRTE7A2 TCR-T cells against antigen-positive tumor cells. KITE-439 TCR-T was used as a control.

FIG. 5 shows the specific killing effect of CRTE7A2 TCR-T cells against antigen-positive tumor cells as determined by IFN-γ secretion assay.

FIG. 6 shows the specific secretion of IFN-γ of CRTE7A2 TCR-T cells against antigen-positive tumor cells. KITE-439 TCR-T was used as a control.

FIG. 7 shows the specific secretion of IFN-γ of CRTE7A2 TCR-T cells against antigen-positive target cells as determined by ELISpot assay. Each group was repeated at least 2 times.

FIG. 8 shows the antigen-specific proliferation of CRTE7A2 TCR-T cells.

FIG. 9 shows the in vivo antitumor activity of CRTE7A2 TCR-T cells against Hela cell transplantation tumors.

### DETAILED DESCRIPTION OF THE INVENTION

Unless otherwise defined, all technical and scientific terms used herein have the same meaning as commonly understood by those skilled in the art. For example, terms used herein are as defined in Janeway C A Jr, Travers P, Walport M et al., Immunobiology, 5th edition, New York: GarlandScience (2001), and Leuenberger, H. G. W, Nagel, B. and Kölbl, H. eds (1995), A multilingual glossary of biotechnological terms: (International Union of Pure and Applied Chemistry (IUPAC) Recommendations), Helvetica Chimica Acta, CH-4010 Basel, Switzerland.

It should be noted that, as used herein and in the appended claims, the singular forms “a”, “an” and “the/the” include plural forms, unless the context clearly defined otherwise. Thus, the terms “a”, “an”, “one or more” and “at least one” can be used interchangeably. Similarly, the terms “comprise”, “include” and “have” can be used interchangeably.

When the term “comprise” is used herein and in the appended claims, it does not exclude other elements. For the purposes of the present disclosure, the term “consist of” is considered to be an embodiment of the term “comprise”. If a group is defined below as including or comprising at least a certain number of embodiments, it is also to be understood as disclosing a group that may consist of only those embodiments.

In one aspect, the present disclosure provides an antigen binding protein comprising a T cell receptor (TCR) alpha chain variable region and a TCR beta chain variable region, wherein the TCR alpha chain variable region comprises a CDR3 having the amino acid sequence: AVISAGTALI (SEQ ID NO:3), or a functional variant thereof formed by insertion, deletion or substitution of one or more amino acids, wherein the antigen binding protein binds to an epitope comprising the amino acid sequence of YMLDLQPET (SEQ ID NO:11) or a complex of the epitope with an MHC molecule.

In another aspect, the present disclosure provides an antigen binding protein comprising a T cell receptor (TCR) alpha chain variable region and a TCR beta chain variable region, wherein the TCR beta chain variable region comprises a CDR3 having the amino acid sequence: ASSLGWRGGLYTEAF (SEQ ID NO:8), or a functional variant thereof formed by insertion, deletion or substitution of one or more amino acids, wherein the antigen binding protein binds to an epitope comprising the amino acid sequence of YMLDLQPET (SEQ ID NO: 11) or a complex of the epitope with an MHC molecule.

In yet another aspect, the present disclosure provides an antigen binding protein comprising a T cell receptor (TCR) alpha chain variable region and a TCR beta chain variable region, wherein the TCR alpha chain variable region comprises a CDR3 having the amino acid sequence: AVISAGTALI (SEQ ID NO:3), or a functional variant thereof formed by insertion, deletion or substitution of one or more amino acids; and the TCR beta chain variable region comprises a CDR3 having the amino acid sequence: ASSLGWRGGLYTEAF (SEQ ID NO:8), or a functional variant thereof formed by insertion, deletion or substitution of one or more amino acids, wherein the antigen binding protein binds to an epitope comprising the amino acid sequence of YMLDLQPET (SEQ ID NO: 11) or a complex of the epitope with an MHC molecule.

As used herein, the term “antigen binding protein” refers to a protein or polypeptide comprising at least one TCR alpha chain CDR3 (CDR3α) and/or at least one TCR beta chain CDR3 (CDR3β) as disclosed herein and capable of binding to an antigenic target HPV E7. Further contemplated herein is an antigen binding protein comprising at least one of CDR1α, CDR2α, CDR1β, CDR2β, alpha chain variable region, beta chain variable region, alpha chain and/or beta chain, or the combination thereof, optionally in combination with other protein domains or portions listed herein.

As used herein, the term “functional variant” refers to a polypeptide that has significant sequence identity to the parent polypeptide and retains the biological activity of the parent polypeptide. A functional variant encompasses, for example, a variant of the polypeptide or protein described herein that retains the ability to specifically bind to the HPV16 E7 antigen to a similar extent to the parent polypeptide, to the same extent as the parent polypeptide, or to a higher extent than the parent polypeptide. The amino acid sequence of the functional variant may, for example, have at least about 50%, 75%, 80%, 90%, 95%, 96%, 97%, 98%, 98.2%, 98.4%, 98.6%, 98.8%, 99%, 99.1%, 99.2%, 99.3%, 99.4%, 99.5%, 99.6%, 99.7%, 99.8%, 99.9% or higher sequence identity to that of the parent polypeptide.

The term “epitope” usually refers to a site on an antigen, usually a (poly) peptide recognized by a binding domain. The term “binding domain” in its broadest sense refers to an “antigen binding site”, i.e. a domain characterizing a molecule that binds to/interacts with a specific epitope on an antigenic target. The antigenic target may comprise a single epitope, but typically comprises at least two epitopes, and depending on the size, conformation and type of the antigen, the antigenic target may include any number of epitopes. The term “epitope” typically includes both linear epitopes and conformational epitopes. A linear epitope is a contiguous epitope contained in a primary sequence of amino acids and typically comprises at least 2 or more amino acids. A conformational epitope is formed by non-contiguous amino acids juxtaposed by folding of a target antigen, in particular, a target (poly) peptide.

In the context of the present disclosure, the term “binding domain” refers in particular to the variable regions of TCR alpha chain and/or beta chain, in particular the CDR3α and CDR3β of the TCR.

As used herein, the term “T cell receptor” or “TCR” includes natural TCRs as well as TCR variants, fragments and constructs. The term thus includes heterodimers as well as multimers and single chain constructs comprising TCR alpha chain and TCR beta chain; optionally comprising other domains and/or portions, provided that the antigen binding protein retains its ability to recognize an antigenic target (for example, its complex with HLA-A*02).

In a natural form, the TCR exists as a complex of several proteins on the surface of T cells. The T cell receptor consists of two (separate) protein chains that are produced by separate T cell receptor alpha and beta (TCRα and TCRβ) genes and are referred to as alpha chain and beta chain. Each chain of the TCR has an N-terminal immunoglobulin-like (Ig)-variable (V) region/domain, an Ig-constant (C) region/domain, a transmembrane/cytomembrane spanning region that anchors the chain into the plasma membrane, and a C-terminal short cytoplasmic tail.

Antigen specificity is conferred by the variable regions of α chain and β chain of TCR. Both variable regions of TCR α and β chains contain three highly variable or complementary determining regions (CDR1α/β, CDR2α/β, and CDR3α/β) surrounded by framework (FR) regions. CDR3 is the major determinant for antigen recognition and specificity (i.e., the ability to recognize and interact with the specific antigen), whereas CDR1 and CDR2 interact primarily with MHC molecules that present antigenic peptides.

The natural TCR recognizes an antigenic peptide that binds to a major histocompatibility complex (MHC) molecule at the surface of the antigen-presenting cell (“presenting/exhibiting on the MHC molecule”). The antigenic peptide presented on the MHC molecule is also referred to herein as a “complex of epitope with MHC molecule”, “epitope-MHC complex” or “target antigenic peptide-MHC complex”. There are two different classes of MHC molecules: MHC I and MHC II, which present peptides from different cellular compartments. MHC class I molecules are expressed on the surface of all nucleated cells in the human and present peptides or protein fragments from intracellular compartments to cytotoxic T cells. In human, MHC is also known as human leukocyte antigen (HLA). There are three main types of MHC class I molecules: HLA-A, HLA-B, and HLA-C. Once the TCR binds to its specific epitope-MHC complex, the T cells are activated and exert a biological effector function.

As will be discussed in detail below, the TCRs provided herein are capable of advantageously (specifically) recognizing an HPV16 E7 antigen, in particular an HPV16 E7₁₁₋₁₉epitope or a complex of the epitope with a MHC molecule, such as a complex of the HPV16 E7₁₁₋₁₉ epitope with HLA-A*02.

In some embodiments of the antigen binding proteins of the present disclosure, the MHC molecule is HLA-A*02 type, such as HLA-A*02:01 type, HLA-A*02:03 type, HLA-A*02:05 type, HLA-A*02:06 type, HLA-A*02:07 type, HLA-A*02:10 type, or HLA-A*02:11 type.

In some embodiments, the TCR alpha chain variable region comprises a CDR3 having the amino acid sequence as shown in SEQ ID NO: 3, and/or the TCR beta chain variable region comprises a CDR3 having the amino acid sequence as shown in SEQ ID NO:8.

As previously described, it is identified that CDR1 and CDR2 of TCRα and β chains are primarily involved in MHC recognition. There is a limited “pool” of CDR1 and CDR2 sequences known to be involved in HLA-A*02-restricted antigen recognition. It is contemplated that the CDR3 domain of the present disclosure can in principle be combined with any one of the CDR1 and CDR2 as shown in SEQ ID NOs: 1-2 and 6-7, provided that the antigen binding protein retains its ability to recognize an antigenic target (for example, its complex with HLA-A*02) to a similar, identical, or even greater extent than the TCR assessed in the appended examples. Available examples of the CDR1 and CDR2 domains include CDR1α comprising or consisting of the sequence as shown in SEQ ID NO: 1, CDR2α comprising or consisting of the sequence as shown in SEQ ID NO: 2, CDR1β comprising or consisting of the sequence as shown in SEQ ID NO: 6, and CDR2β comprising or consisting of the sequence as shown in SEQ ID NO: 7.

In some embodiments, the TCR alpha chain variable region comprises CDR1, CDR2, and CDR3 having the amino acid sequences as shown in SEQ ID NO:1, SEQ ID NO:2, and SEQ ID NO: 3, respectively, or a functional variant thereof formed by insertion, deletion, or substitution of one or more amino acids; and/or the TCR beta chain variable region comprises a beta chain CDR1, CDR2, and CDR3 having the amino acid sequences as shown in SEQ ID NO:6, SEQ ID NO: 7 and SEQ ID NO:8, respectively, or a functional variant thereof formed by insertion, deletion or substitution of one or more amino acids.

In another aspect, the present disclosure provides an antigen binding protein comprising a T cell receptor (TCR) alpha chain variable region and a TCR beta chain variable region, wherein the TCR alpha chain variable region comprises CDR1, CDR2, and CDR3 having amino acid sequences as shown in SEQ ID NO:1, SEQ ID NO:2, and SEQ ID NO:3, respectively; and/or the TCR beta chain variable region comprises the beta chain CDR1, CDR2, and CDR3 having the amino acid sequences as shown in SEQ ID NO:6, SEQ ID NO:7, and SEQ ID NO:8, respectively. In some embodiments, the TCR alpha chain variable region comprises CDR1, CDR2, and CDR3 having the amino acid sequences as shown in SEQ ID NO:1, SEQ ID NO:2, and SEQ ID NO:3, respectively; and the TCR beta chain variable region comprises beta chain CDR1, CDR2, and CDR3 having the amino acid sequences as shown in SEQ ID NO:6, SEQ ID NO: 7, and SEQ ID NO:8, respectively.

In some embodiments of the antigen binding protein of the present disclosure, the TCR alpha chain variable region comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:4.

In some embodiments of the antigen binding protein of the present disclosure, the TCR beta chain variable region comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:9.

As used herein, the term “sequence identity” indicates the extent to which two (nucleotide or amino acid) sequences have the same residues at the same positions in alignment, and is typically expressed as a percentage. In an embodiment, identity is determined over the overall length of the sequences being compared. Thus, two copies with the identical sequence have 100% identity, while sequences that are less highly conserved and have deletions, additions or substitutions may have a lower degree of identity. Those skilled in the art will recognize that a number of algorithms can be used to determine sequence identity using standard parameters, such as Blast (Altschul et al. (1997) Nucleic Acids Res. 25: 3389-3402), Blast2 (Altschul et al. (1990) J. Mol. Biol. 215: 403-410), Smith-Waterman (Smith et al. (1981) J. Mol. Biol. 147: 195-197), and ClustalW.

Thus, the amino acid sequence of SEQ ID NO: 4 or 9 may, for example, be a “subject sequence” or a “reference sequence”, while a different amino acid sequence of TCR alpha or beta chain variable region may be a “query sequence”.

In some embodiments, the TCR alpha chain variable region comprises the amino acid sequence as shown in SEQ ID NO:4. In some embodiments, the TCR β chain variable region comprises the amino acid sequence as shown in SEQ ID NO:9.

In some embodiments, the TCR alpha chain variable region is contained on a first polypeptide and the TCR β chain variable region is contained on a different second polypeptide. In some embodiments, the TCR alpha chain variable region and the TCR beta chain variable region are contained on a single polypeptide.

In some embodiments, the antigen binding protein is soluble or membrane-bound.

The antigen binding protein of the present disclosure may be provided in a soluble form, for example in the form of a soluble TCR. Soluble TCR (sTCR) can be used as a diagnostic tool and a vector or “adaptor” for specifically targeting therapeutic agents or effector cells to cancer cells, for example, expressing antigenic targets recognized by the soluble TCR. The soluble TCR is typically a fragment or construct comprising a TCR alpha and/or beta chain or a variable region or CDR thereof, and optionally is stabilized by a disulfide bond or covalently linked by a suitable linker. Typically, the soluble TCR does not include, for example, a transmembrane region.

The antigen binding protein of the present disclosure may also be provided in a membrane-bound form, for example in the form of a membrane-bound TCR. Typically, the membrane-bound TCR includes a transmembrane region to anchor it to the cell membrane.

In some embodiments, the antigen binding protein is selected from a TCR, a chimeric antigen receptor (CAR), a Fc polypeptide, or antigen-binding fragments thereof.

In some embodiments, the antigen binding protein is a TCR or an antigen-binding fragment thereof, and the antigen binding protein further comprises a TCR constant region or a fragment thereof.

As used herein, the term “constant region” may be a human constant region or derived from another species, resulting in a “chimeric” TCR. For example, human α chain and/or β chain may be replaced by their murine counterparts (“murine-derived”), and the murine counterparts have been found to enhance surface expression of the human TCR and enhance binding stability of the human TCR to CD3 co-receptors by supporting preferential pairing of TCR α and β chains.

In some embodiments, the TCR constant region is a murine constant region or a human constant region.

It is reported that the addition of disulfide bonds to the constant region may promote the correct pairing of TCR α and β chains (Kuball J et al. Blood. 2007 Mar. 15; 109(6): 2331-8). Therefore, the present disclosure also envisages the addition of one or more cysteine modifications to the constant region to form disulfide bonds between the TCR α and TCR β chains.

In some embodiments, the TCR constant region comprises a TCR alpha chain constant region and/or a TCR beta chain constant region. In an embodiment, the TCR alpha chain constant region and/or TCR beta chain constant region comprises at least one cysteine mutation relative to a wild-type sequence to form a disulfide bond between the TCR alpha chain and the TCR beta chain.

In some embodiments, the cysteine mutation is at one or more of the following positions: position 48 of the wild-type human TCR alpha chain constant region, position 48 of the wild-type murine TCR alpha chain constant region, position 57 of the wild-type human TCR beta chain constant region, and position 57 of the wild-type murine TCR beta chain constant region.

The sequence of the wild-type TCR constant region can be found in the public database of the International Immunogenetics Information System (IMGT). For example, the sequence of the TCR alpha chain constant domain is “TRAC*01” and the sequence of TCR beta chain constant domain is “TRBC1*01” or “TRBC2*01”.

To facilitate the description of the location of the cysteine mutation, the positions of the amino acid sequences of the wild-type TCR constant region in the present disclosure are numbered according to the nomenclature of the International Immunogenetics Information System (IMGT). For example, if an amino acid in the TCR alpha chain constant region (TRAC) is designated with a position number of 48 in the IMGT, it is described herein as the amino acid at position 48 of the TCR alpha chain constant region (TRAC); if an amino acid in the TCR beta chain constant region (TRBC) is designated with a position number of 57 in the IMGT, it is described herein as the amino acid at position 57 of the TCR beta chain constant region (TRBC); and so on. Herein, the position numbering of the amino acid sequences of the variable regions TRAV and TRBV is based on the position numbering listed in IMGT. If an amino acid in TRAV is designated with a position number of 46 in IMGT, it is described herein as the amino acid at position 46 of TRAV; and so on. When the sequence position numbering of other amino acids is specifically described in the present disclosure, they are numbered as specifically described.

In some embodiments, the TCR alpha chain constant region further comprises an LVL mutation or an LIV mutation such that the constant region (and/or the transmembrane region) comprises the amino acid sequence LLVIVLRIL. For example, when the TCR alpha chain comprises a human constant region, the human constant region may comprise an LVL mutation such that the constant region (and/or the transmembrane region) comprises the amino acid sequence LLVIVLRIL. When the TCR alpha chain contains a murine constant region, the murine constant region may comprise an LIV mutation such that the constant region (and/or the transmembrane region) comprises the amino acid sequence LLVIVLRIL.

In some embodiments, the TCR alpha chain constant region comprises an amino acid sequence having at least 80%, at least 85%, at least 90% at least 91%, at least 92%, at least 93%, at least 94%, at least 95%, at least 96%, at least 97%, at least 98%, at least 99%, or 100% sequence identity to any one of SEQ ID NOs: 14-19, and/or the TCR β chain constant region comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 21-30.

In some embodiments, the TCR alpha chain constant region comprises the amino acid sequence as shown in SEQ ID NO:19 and the TCR beta chain constant region comprises the amino acid sequence as shown in SEQ ID NO:26.

In some embodiments, the fragment of the TCR constant region is an extracellular segment of the TCR constant region.

In some embodiments, the antigen binding protein is a TCR comprising an alpha chain and a beta chain. In some embodiments, the TCR alpha chain and/or beta chain may comprise a leader sequence. For example, the leader sequence of the TCR alpha chain can have the amino acid sequence as shown in SEQ ID NO: 12 (MISLRVLLVILWLQLSWVWSQ). The leader sequence of the TCR beta chain can have the amino acid sequence as shown in SEQ ID NO:13 (MGPGLLCWALLCLLGAGLV). The leader sequence of the TCR alpha chain can be encoded by the nucleotide sequence as shown in SEQ ID NO:48 (ATGATATCCTTGAGAGTTTTACTGGTGATCCTGTGGCTTCAGTTAAGCTGGGTTTGG AGCCAA). The leader sequence of the TCR β chain can be encoded by the nucleotide sequence as shown in SEQ ID NO:49 (ATGGGCCCCGGGCTCCTCTGCTGGGCACTGCTTTGTCTCCTGGGAGCAGGCTTAGTG).

In some embodiments, the antigen binding protein further comprises a transmembrane region and/or a cytoplasmic region.

In some embodiments, the antigen binding protein comprises a TCR alpha chain comprising an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to any one selected from SEQ ID NOs: 32-37; and/or a TCR beta chain comprising an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to any one selected from SEQ ID NOs: 38-47.

In some embodiments, the antigen binding protein further comprises an intracellular signaling region. In some embodiments, the antigen binding protein further comprises one or more antigen-binding regions that bind other antigens or epitopes.

In some embodiments, the antigen binding protein is isolated or purified.

As used herein, the term “isolated or purified” means that the “isolated or purified” antigen binding protein has been identified, isolated and/or recovered from components of the environment in which it is produced, such that the “isolated or purified” antigen binding protein is free or substantially free of other contaminant components from the environment in which it is produced that may interfere with its therapeutic or diagnostic use. Contaminant components may include enzymes, hormones, and other proteins or non-protein solutes. Thus, the “isolated or purified” antigen binding protein may be prepared by at least one purification that removes or substantially removes these contaminant components. In yet another aspect, the present disclosure provides a nucleic acid encoding the antigen binding protein of the present disclosure.

In some embodiments, the nucleic acid comprises a nucleotide sequence having at least 80%, at least 85%, at least 90%, at least 95%, or 100% sequence identity to SEQ ID NO:5, and/or a nucleotide sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 10. In some embodiments, the nucleic acid further comprises a nucleotide sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:20, and/or a nucleotide sequence having at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 31.

In another aspect, the present disclosure provides a vector comprising the nucleic acid of the present disclosure.

As used herein, the term “vector” is a nucleic acid molecule used as a vehicle for transferring (exogenous) genetic materials into a host cell in which the nucleic acid molecule as a vector can, for example, be replicated and/or expressed. The term “vector” encompasses, but is not limited to, plasmids, viral vectors (including retroviral vectors, lentiviral vectors, adenoviral vectors, cowpox virus vectors, polyomavirus vectors and adenovirus-associated vectors (AAVs)), phages, phasmids, cosmids and artificial chromosomes (including bacterial artificial chromosomes (BACs) and yeast artificial chromosomes (YACs)). The vector itself is usually a nucleotide sequence, usually comprises a DNA sequence comprising an insert (transgene) and a larger sequence that serves as the “backbone” of the vector. The engineered vector usually comprises an origin for self-replication in the host cell (if stable expression of polynucleotides is desired), a selection marker and a restriction enzyme cleavage site (e.g., a polyclonal site, MCS). The vector may additionally comprise a promoter, a genetic marker, a reporter gene, a targeting sequence, and/or a protein purification tag. As known to those skilled in the art, a large number of suitable vectors are known to those skilled in the art, and many are commercially available. Examples of suitable vectors are provided in J. Sambrook et al, Molecular Cloning: a Laboratory Manual (4th ed.), Cold Spring Harbor Laboratory, Cold Spring Harbor Laboratory Press, New York (2012), which is incorporated herein by reference in their entirety.

In some embodiments, the vector may be selected from the group consisting of lentiviral vectors, retroviral vectors, plasmids, and DNA vectors, mRNA vectors, transposon-based vectors, and artificial chromosomes.

In yet another aspect, the present disclosure provides a cell comprising the antigen binding protein, the nucleic acid, or the vector according to the present disclosure.

As used herein, the term “cell” refers to any type of cell capable of expressing the antigen binding protein of the present disclosure. The cell may be a eukaryotic cell, e.g., a vegetation (which does not have the potential to develop into a plant), animal, fungus, or algae cell, or may be a prokaryotic cell, e.g., a bacterium or protozoan cell. The cell may be a cultured cell or a primary cell, i.e. directly isolated from an organism, e.g. a human. The cell may be an adherent cell or suspension cell, i.e. cell grown in suspension. Suitable host cell is known in the art and includes, for example, a DH5α E. coli cell, Chinese hamster ovary cell, monkey VERO cell, COS cell, HEK293 cell, and the like. For the purpose of producing the antigen binding protein of the present disclosure, the cell may be a mammalian cell. In an embodiment, the host cell is a human cell.

In some embodiments, the cell is selected from the group consisting of lymphocytes (e.g., T cells, NK cells), monocytes (e.g., PBMCs), and stem cells. As used herein, the term “stem cell” is a stem cell used for expressing the antigen binding protein (particularly the TCR) of the present disclosure. For example, a stem cell may be a lymphoid progenitor cell, an induced pluripotent stem cell (iPSC), or a hematopoietic stem cell (HSC). In some embodiments, the stem cell does not include embryonic stem cells obtained by destroying a human embryo, and/or does not include totipotent stem cells that are used to develop and form an individual animal. Transferring a gene to stem cells does not typically result in expression of a TCR on the surface of the cells, as stem cells does not express CD3 molecule on the surface. However, when stem cells differentiate into lymphoid precursors that migrate to thymus, expression of CD3 molecules will initiate expression of the introduced TCR molecules on the surface of thymocytes.

In some embodiments, the stem cell is a lymphoid progenitor cell or an induced pluripotent stem cell (iPSC).

In some embodiments, the cell is a T cell. The T cell may be any T cell, such as a cultured T cell, e.g., a primary T cell or a T cell from a cultured T cell line, e.g., Jurkat, SupTl, etc., or a T cell obtained from a mammal. If obtained from a mammal, the T cell may be obtained from many sources including, but not limited to, blood, bone marrow, lymph node, thymus or other tissues or fluids. The T cell may also be enriched or purified. In an embodiment, the T cell is a human T cell. In an embodiment, the T cell is a T cell isolated from human. The T cell may be any type of T cells and may be at any developmental stage, including but not limited to CD4+/CD8+ double positive T cells, CD4+ helper T cells, such as Th1 and Th2 cells, CD4+ T cells, CD8+ T cells (e.g., cytotoxic T cells), tumor infiltrating lymphocytes (TILs), memory T cells (e.g., central memory T cells and effector memory T cells), naive T cells, and the like. In some embodiments, the T cell does not express an endogenous TCR.

It is contemplated that effector cells expressing the antigen binding protein (such as the TCR) as described herein bind to the antigenic target thereof (such as the HPV16 E7₁₁₋₁₉ epitope presented by antigen-presenting cells on HLA-A*02) with high affinity. The term “affinity” or “binding affinity” refers to the ability of the cells expressing the antigen binding protein (in particular, the T cells expressing the TCR as described herein) to respond to the ligand at given concentration in vitro, and the ability is considered to correlate with the in vivo effector capacity of the cells expressing the antigen binding protein (e.g. the TCR). By definition, the cells expressing the antigen binding protein (e.g., the TCR) having high binding affinity respond to very low doses of antigen in vitro, whereas such cells having lower binding affinity require higher amounts of antigen if the immune response similar to that of the cells expressing the antigen binding proteins (e.g., the TCR) having high affinity is desired. Thus, binding affinity can be regarded as a quantitative determinant of the activation threshold of the cells expressing the antigen binding protein (e.g., the TCR). This is measured by exposing such cells to different amounts of homologues antigen in vitro. The cells expressing the antigen binding protein (e.g., the TCR) having high binding affinity respond to low doses of antigen. For example, if TCR-expressing cells secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, or 20,000 pg/mL or more) interferon gamma (IFN-y) when co-cultured with antigen-negative HLA-*02-expressing target cells, the TCR-expressing cells are generally considered as binding to the antigen target thereof with “high” binding affinity.

In another aspect, the present disclosure provides a method of preparing the cells of the present disclosure, comprising transducing or transfecting the cells with the vector of the present disclosure.

As used herein, the term “transfection” is a process of intentionally introducing nucleic acid molecules or polynucleotides (including vectors) into target cells. One example is RNA transfection, that is, a process of introducing RNA (e.g., in vitro transcribed RNA, ivtRNA) into host cells. This term is primarily used for non-viral methods in eukaryotic cells. The term “transduction” is commonly used to describe virus-mediated transfer of nucleic acid molecules or polynucleotides. Transfection of animal cells typically involves the opening of transient pores or “holes” in the cell membrane to allow uptake of the materials. Transfection can be performed by using calcium phosphate, electroporation, cell extrusion, or by mixing cationic lipids with the materials to produce liposomes that are fused with the cell membrane and deposit their cargos into the interior. Exemplary techniques for transfecting eukaryotic host cells include lipid vesicle-mediated uptake, heat shock-mediated uptake, calcium phosphate-mediated transfection (calcium phosphate/DNA co-precipitation), microinjection, and electroporation.

In some embodiments, the method further comprises amplifying and/or activating the cells prior to or after the transduction or transfection.

In another aspect, the present disclosure provides a conjugate comprising the antigen binding protein of the present disclosure and an active agent coupled or conjugated to the antigen binding protein.

In some embodiments, the active agent is selected from the group consisting of detectable markers, immunostimulatory molecules, and therapeutic agents. In an embodiment, the detectable marker is selected from the group consisting of biotins, streptavidin, enzymes or catalytically active fragments thereof, radionuclides, nanoparticles, paramagnetic metal ions, nucleic acid probes, contrast agents, and fluorogenic, phosphorescent or chemiluminescent molecules. In an embodiment, the immunostimulatory molecule is selected from the group consisting of cytokines (e.g. interleukin-2 (IL-2) and interferon gamma (IFN-γ)), chemokines (e.g. interleukin-8 (IL-8)), platelet factors (e.g. platelet factor 4), and complement initiators. In an embodiment, the therapeutic agent is selected from the group consisting of immunomodulators, radioactive compounds, enzymes, chemotherapeutic agents and toxins. Other suitable therapeutic agents include small molecule cytotoxic agents, i.e. compounds having the ability to kill mammalian cells having a molecular weight of less than 700 daltons. Such compounds may also contain toxic metals having cytotoxic effects. In addition, these small molecule cytotoxic agents also include precursor drugs, i.e. compounds that decay or are transformed under physiological conditions to release cytotoxic agents. Examples of such agents include cisplatin, maytenin derivatives, rachelmycin, calicheamicin, docetaxel, etoposide, gemcitabine, isocyclic phosphorylamine, irinotecan, melphalan, mitoxantrone, sorfimer sodiumphotofrin II, temozolomide, topotecan, trimetreate glucuronate, auristatin E, vincristine, and doxorubicin; peptide cytotoxin, i.e., proteins or the fragments thereof having the ability to kill mammalian cells; e.g., ricin, diphtheria toxin, Pseudomonas bacterial exotoxin A, Dnase, and RNase; radionuclides, i.e., unstable isotopes of elements that emit one or more alpha or beta particles or gamma rays while decaying, e.g., iodine-131, rhenium-186, indium-111, yttrium-90, bismuth-210 and bismuth-213, actinium-225, and astatine-213; chelating agents which can be used to facilitate the binding of these radionuclides to molecules or multimers thereof; or heterologous protein domains, homologous protein domains, viral/bacterial protein domains, viral/bacterial peptides.

In another aspect, the present disclosure provides a composition comprising the antigen binding protein, the nucleic acid, the vector, or the cell of the present disclosure. In an embodiment, the composition further comprises a pharmaceutically acceptable carrier or excipient.

The term “composition” refers in particular to a composition suitable for the administration to humans. However, the term generally also encompasses a composition suitable for the administration to non-human animals. The composition and its components (i.e. active agents and optionally carriers or excipients) may be pharmaceutically acceptable, i.e. capable of eliciting the desired therapeutic effect without causing any undesired local or systemic effects in the recipient. The pharmaceutically acceptable compositions of the present disclosure may be, for example, sterile. Specifically, the term “pharmaceutically acceptable” may indicate approval by a regulatory agency or other recognized pharmacopoeia for use in animals, and more particularly in humans.

The term “excipient” includes fillers, adhesives, disintegrants, coating agents, adsorbents, anti-adhesives, flow aids, preservatives, antioxidants, flavoring agents, coloring agents, sweeteners, solvents, co-solvents, buffers, chelating agents, viscosity imparting agents, surfactants, diluents, wetting agents, carriers, diluents, preservatives, emulsifiers, stabilizers and tension regulators. It is known to those skilled in the art to select suitable excipients to prepare the composition of the present disclosure. Exemplary carriers for use in the composition of the present disclosure include saline, buffered saline, glucose, and water. Typically, the selection of a suitable excipient depends in particular on the active agent used, the disease to be treated and the desired dosage form of the composition.

Depending on the active agent employed (e.g. soluble TCR), the composition of the present disclosure may be formulated to various forms, such as solid, liquid, gaseous or lyophilized forms, and in particular may be in the form of ointments, creams, transdermal patches, gels, powders, tablets, solutions, aerosols, granules, pills, suspensions, emulsions, capsules, syrups, liquids, elixirs, infusions, tinctures or fluid extracts, or a form particularly suitable for the desired administration method. The processes of pharmaceutical production known to the present disclosure are shown in the 22nd edition of Remington's Pharmaceutical Sciences (Ed. MaackPublishing Co, Easton, Pa., 2012) and may include, for example, the conventional processes of mixing, lysing, granulating, sugar-coating, grinding, emulsifying, encapsulating, embedding, or lyophilizing. Compositions comprising, for example, the host cell or the soluble TCR as described herein are typically provided in liquid form and may comprise pharmaceutically acceptable buffering agents.

In some embodiments, the composition of the present disclosure further comprises a second therapeutic agent. In an embodiment, the second therapeutic agent is selected from the group consisting of antibodies, chemotherapeutic agents, and small molecule drugs.

Examples of the second therapeutic agent include known anti-cancer drugs such as cisplatin, maytenin derivatives, rachelmycin, calicheamicin, docetaxel, etoposide, gemcitabine, isocyclic phosphorylamine, irinotecan, melphalan, mitoxantrone, sorfimer sodiumphotofrin II, temozolomide, topotecan, trimetreate glucuronate, auristatin E, vincristine, and doxorubicin; and peptide cytotoxin, such as ricin, diphtheria toxin, Pseudomonas bacterial exotoxin A, Dnase, and RNase; radionuclides, e.g. iodine 131, rhenium 186, indium 111, iridium 90, bismuth 210 and 213, actinium 225 and astatine 213; prodrugs, e.g. antibody-directed enzyme prodrugs; immunostimulants, e.g. IL-2, chemokines e.g. IL-8, platelet factor 4; antibodies or fragments thereof, e.g. anti-CD3 antibodies or fragments thereof; complement initiators; heterologous protein domains, homologous protein domains, viral/bacterial protein domains, and viral/bacterial peptides.

In yet another aspect, the present disclosure provides a method of treating or preventing an HPV-positive disease in a subject comprising administering to the subject an effective amount of the antigen binding protein of the present disclosure.

In another aspect, the present disclosure provides a method of treating or preventing an HPV-positive disease in a subject comprising administering to the subject an effective amount of the cell of the present disclosure.

As used herein, the term “treatment”, “treat” or “treating” includes therapeutic or prophylactic treatment in a subject in need thereof. The term “therapeutic or prophylactic treatment” includes prophylactic treatment intended to completely prevent clinical and/or pathological manifestations or therapeutic treatment intended to improve or alleviate clinical and/or pathological manifestations. Thus, the term “treatment” also includes improvement or prevention of diseases.

As used herein, the term “effective amount” refers to an amount of a therapeutic agent sufficient to achieve such treatment or prevention when administered to a subject for the treatment or prevention of a disease. The “effective amount” may vary depending on the compound, the disease and its severity, and the age, weight, etc. of the subject to be treated. A “therapeutically effective amount” refers to an effective amount for the therapeutic treatment. A “prophylactic effective amount” refers to an effective amount for prophylactic treatment.

Therapeutic efficacy and toxicity can be determined by standard procedures such as ED50 (a dose that is therapeutically effective in 50% of the population) and LD50 (a dose that is lethal to 50% of the population) in such as cell cultures or experimental animals. The dose ratio between therapeutic and toxic effects is a therapeutic index and can be expressed as a ratio of ED50/LD50. The pharmaceutical composition may exhibits a large therapeutic index.

The exact dose of the antigen binding protein or cell administered can be determined by those skilled in the art using known techniques. A suitable dose provides a sufficient amount of the active agent of the disclosure and may be therapeutically effective, i.e., sufficient to elicit, for example, a therapeutic or prophylactic response in a subject or animal within a reasonable time period. For example, the dose of the antigen binding protein of the present disclosure such as TCR should be sufficient to bind to a cancer antigen or detect, treat, or prevent a cancer within a time period of about 2 hours or more, e.g., 12 hours to 24 hours or more from the time of administration (e.g., 1 month, 2 months, 3 months, 6 months, 12 months, 24 months, etc.). In some embodiments, the time period may even be longer. As known in the art, adjustments may be necessary depending on therapeutic purpose (such as alleviation of the acute onset of a disease), dosing route, time and frequency, time and frequency for administrating the formulation, age, weight, general health condition, gender, diet, severity of disease condition, drug combination, response sensitivity and tolerance/response to treatment.

A number of assays for determining the dose to be administered are known in the art. For the purposes of the present disclosure, the assays, including after administration of a given dose of T cells expressing the antigen binding protein (e.g., TCR) of the present disclosure to a group of mammals (each being administered a different dose of T cells), comparing the extent of target cell lysis or IFN-γ secretion achieved by such T cells, may be used to determine a starting dose to be administered to a mammal. The extent of target cell lysis or IFN-γ secretion achieved after administration of a given dose can be determined by methods known in the art. The dose of the antigen binding protein or the cell of the present disclosure is also determined by the presence, nature and extent of any adverse side effects that may accompany the administration of the antigen binding protein or the cell of the present disclosure. Typically, the dose of the antigen binding protein or the cell of the present disclosure to be administered to each individual patient is determined by the attending physician, in consideration of a variety of factors such as age, weight, general health, diet, gender, active agent to be administered, route of administration, and severity of the treated medical condition. In some embodiments of the treatment method of the present disclosure, the number of cells administered per infusion may vary, for example, from about 1×10⁶ to about 1×10¹² cells or more. In some embodiments, less than 1×10⁶ cells may be administered.

It should be recognized that treatment may require a single administration or multiple administrations of a therapeutically effective dose of the active agent of the disclosure. For example, depending on the formulation, half-life and clearance of a particular composition, some compositions may be administered every 3 to 4 days, weekly, or every two weeks, or administered once over a period of one month.

The composition of the present disclosure may be suitable for the administration by a variety of routes. Typically, administration is accomplished parenterally. Parenteral delivery methods include topical, intra-arterial, intramuscular, subcutaneous, intramedullary, intrathecal, intracardiac, intravenous, intraperitoneal, intrauterine, intravaginal, sublingual, or intranasal administration.

The terms “subject” or “individual” or “animal” or “patient” are used interchangeably herein and refer to any subject in need of treatment, in particular mammalian subjects. In general, mammalian subjects include humans, non-human primates, dogs, cats, guinea pigs, rabbits, rats, mice, horses, cows, dairy cattle, etc. However, it is particularly contemplated that the TCRs, nucleic acids, vectors, host cells, and pharmaceutical compositions provided herein are used to treat human subjects, particularly those are HLA-A*02 positive, such as HLA-A*02:01, HLA-A*02:03, HLA-A*02:05, HLA-A*02:06, HLA-A*02:07 HLA-A*02:10 or HLA-A*02:11 positive.

In some embodiments of the treatment method of the present disclosure, the cells are autologous or allogeneic to the subject.

In some embodiments, the method comprises: (i) isolating a sample containing cells from the subject; (ii) transducing or transfecting the cells with the vector of the present disclosure; and (iii) administering the cells obtained in item (ii) to the subject. In some embodiments, the method further comprises knocking out an endogenous TCR in the cells after item (i) and prior to item (ii). In some embodiments, the method further comprises administering a second therapeutic agent. In an embodiment, the second therapeutic agent is selected from the group consisting of antibodies, chemotherapeutic agents and small molecule drugs. Examples of the second therapeutic agent are as described above.

In some embodiments of the treatment method of present disclosure, the HPV-positive disease is selected from the group consisting of an HPV infection, an HPV premalignancy, and an HPV cancer. The cancer may be any cancer, including acute lymphocytic cancer, acute myeloid leukemia, alveolar rhabdomyosarcoma, bone cancer, brain cancer, breast cancer, anal cancer, anal canal cancer or rectal anal cancer, eye cancer, intrahepatic cholangiocarcinoma, joint cancer, neck cancer, gallbladder cancer or pleural cancer, nasal cancer, nasal cavity cancer or middle ear cancer, oral cancer, vaginal cancer, vulvar cancer, chronic lymphocytic leukemia, chronic myeloid cancer, colon cancer, esophageal cancer, cervical cancer, gastrointestinal carcinoid tumors, glioma, Hodgkin's lymphoma, hypopharyngeal cancer, kidney cancer, laryngeal cancer, liver cancer, lung cancer, malignant mesothelioma, melanoma, multiple myeloma, nasopharyngeal carcinoma, non-Hodgkin's lymphoma, oropharyngeal cancer, ovarian cancer, penile cancer, pancreatic cancer, peritoneal cancer, omental cancer and mesenteric cancer, pharyngeal cancer, prostate cancer, rectal cancer, kidney cancer, skin cancer, small bowel cancer, soft tissue cancer, stomach cancer, testicular cancer, thyroid cancer, uterine cancer, ureteral cancer, and bladder cancer. Cancers may be HPV16-positive cancers. While the cancers most commonly associated with HPV16 infection include cervical cancer, oropharyngeal cancer, anal cancer, anal canal cancer, recto-anal cancer, vaginal cancer, vulvar cancer, and penile cancer, the method of the present disclosure can be used to treat any HPV16 positive cancers, including those present in other anatomical regions. In an embodiment, the cancer is selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

In another aspect, the present disclosure provides a method of detecting (e.g., diagnosing) an HPV positive disease in a subject, wherein the method comprises (i) contacting a sample obtained from the subject with the antigen binding protein, cell, or conjugate of the present disclosure; and (ii) detecting the presence of an HPV antigen in the sample, wherein the presence of the HPV antigen is indicative of the HPV positive disease. The HPV positive disease may be selected, for example, from the group consisting of an HPV infection, an HPV premalignancy and an HPV cancer. The cancer is as defined above.

In some embodiments, the sample obtained from the subject may be a blood sample, a urine sample, a tissue sample, or a cell sample. In some embodiments, the method is performed in vitro. In some embodiments, the method comprises (i) contacting the sample obtained from the subject with the conjugate of the present disclosure, wherein the conjugate comprises a detectable marker; and (ii) detecting the presence of an HPV antigen in the sample by detecting the detectable marker. Examples of the detectable marker include, but are not limited to, biotins, streptavidin, enzymes or catalytically active fragments thereof, radionuclides, nanoparticles, paramagnetic metal ions, nucleic acid probes, contrast agents, and fluorogenic, phosphorescent, or chemiluminescent molecules. In an embodiment, examples of the detectable marker include, but are not limited to enzymes or catalytically active fragments thereof, radionuclides, fluorogenic, phosphorescent, or chemiluminescent molecules.

In yet another aspect, the present disclosure provides a kit for detecting the presence of a positive epitope in a sample to be tested comprising the antigen binding protein or the conjugate according to the present disclosure, wherein the epitope is an epitope comprising YMLDLQPET (SEQ ID NO:11).

In some embodiments, the kit is a kit for detecting (e.g., diagnosing) an HPV-positive disease in a subject comprising the antigen binding protein or the conjugate of the present disclosure. The HPV-positive condition may, for example, be selected from the group consisting of an HPV infection, an HPV premalignancy, and an HPV cancer. The cancer is as defined above.

In some embodiments, the conjugate comprises a detectable marker. Examples of the detectable marker include, but are not limited to, biotins, streptavidin, enzymes or catalytically active fragments thereof, radionuclides, nanoparticles, paramagnetic metal ions, nucleic acid probes, contrast agents, and fluorogenic, phosphorescent, or chemiluminescent molecules. In an embodiment, examples of the detectable marker include, but are not limited to, enzymes or catalytically active fragments thereof, radionuclides, fluorogenic, phosphorescent, or chemiluminescent molecules. In some embodiments, the kit may further comprise instructions on how to use the kit.

In another aspect, the present disclosure provides use of the antigen binding protein, the nucleic acid, the vector, the cell, or the composition of the present disclosure in the preparation of a medicament for the treatment or prevention of an HPV-positive disease in a subject.

In yet another aspect, the present disclosure provides the antigen binding protein, the nucleic acid, the vector, the cell, or the composition of the present disclosure for use in treating or preventing an HPV-positive disease in a subject.

In another aspect, the present disclosure provides use of the antigen binding proteins or the conjugate of the present disclosure in the preparation of a kit for detecting the presence of a positive epitope in a sample to be tested, wherein the epitope is an epitope comprising YMLDLQPET (SEQ ID NO:11).

In some embodiments, the present disclosure provides use of the antigen binding protein or the conjugate of the present disclosure in the preparation of a kit for detecting (e.g. diagnosing) an HPV-positive disease in a subject.

In yet another aspect, the present disclosure provides the antigen binding protein or the conjugate of the present disclosure for use in detecting the presence of a positive epitope in a sample to be tested, wherein the epitope is an epitope comprising YMLDLQPET (SEQ ID NO: 11), for example, for use in detecting (e.g., diagnosing) an HPV-positive disease in a subject.

In some embodiments of the uses of the present disclosure, the subject has an HLA-A*02:01 allele, such as an HLA-A*02:01, HLA-A*02:03, HLA-A*02:05, HLA-A*02:06, HLA-A*02:07, HLA-A*02:10, or HLA-A*02:11 allele. In some embodiments, the HPV-positive disease is selected from the group consisting of an HPV infection, an HPV premalignancy and an HPV cancer. The cancer may be selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

The present invention is further elaborated by the following specific examples. These examples are intended to illustrate the invention only and are not intended to limit the scope of the invention. Experimental methods for which specific conditions are not indicated in the following examples are performed through conventional conditions in the art, e.g., those described in Sambrook and Russeii et al, Molecular Cloning: a Laboratory Manual (Third Edition) (2001), CSHL Press, or through those as recommended by the manufacturer. Unless otherwise stated, the experimental materials and reagents used in the following examples are commercially available.

### EXAMPLE 1

### Screening for HPV16 E7-Specific TCRs

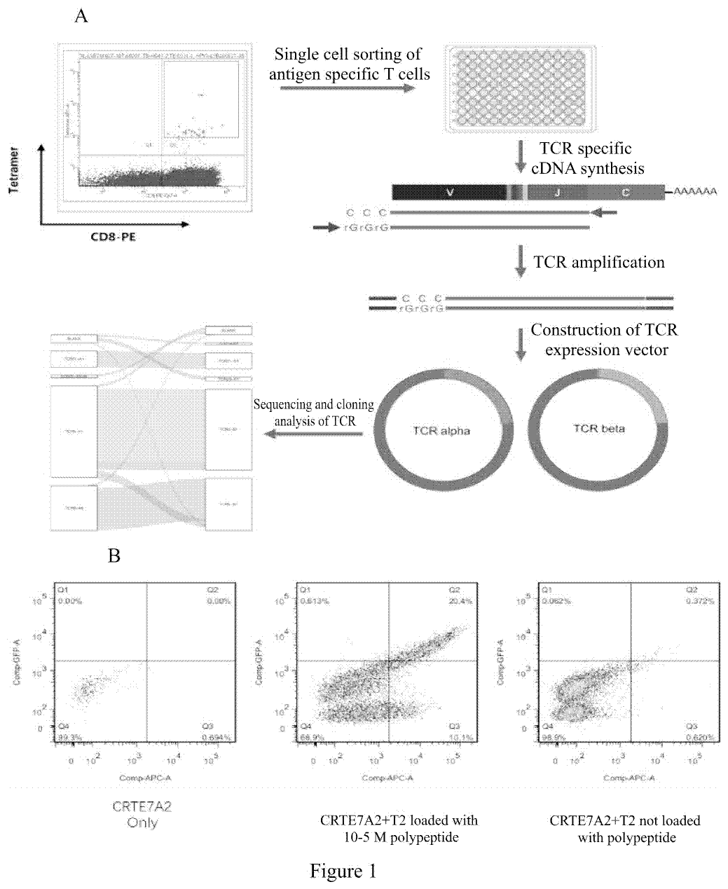

After obtaining ethical approval and informed consent from patients, post-operative tumor tissues from patients with HPV16 positive cervical cancer (HLA-A*02:01) were obtained and tumor infiltrating lymphocytes (TILs) were cultured and amplified from the tumor tissues. After adequate amplification of TILs, CD8 and HLA-A*02: 01/HPV16 E7₁₁₋₁₉ tetramer strongly-stained positive cells were sorted using flow cytometry. The mRNA was extracted from the sorted CD8+/tetramer+ cells, reverse transcribed and amplified to obtain the TCR α chain and β chain variable region (V region) genes. The obtained TCR α chain and β chain V region genes were further constructed into a lentiviral vector containing the TCR constant region (C region) gene. The cloned vector could express the complete TCR α chain and β chain, respectively (FIG. 1A).

Various candidate TCR pairs were introduced into the established T cell reporter cell lines, and the antigenic specificity and affinity of the TCR pairs were confirmed by an antigen-specific activation assay. Using the above cloning and confirmation strategies, a TCR, referred to as CRTE7A2, specifically recognizing the HPV16 E711-19 epitope (YMLDLQPET, SEQ ID NO:11) presented by HLA-A*02:01 was obtained (FIG. 1B).

The amino acid sequences of the variable regions of CRTE7A2 and their coding sequences are as shown in Tables 1 and 2, respectively, and the CDR sequences of the α chain and β chain variable regions are as shown in Table 3. The sequences of the constant regions of the recombinant TCR are as shown in Table 4. The amino acid sequences of the α chain and β chain of the recombinant TCR are as shown in Table 5.

TABLE 1
Amino acid sequences of the variable
regions of CRTE7A2

| | |
|---|---|
| α | RKEVEQDPGPFNVPEGATVAFNCTYSNSASQSFFWYRQDCRKEP |
| chain | KLLMSVYSSGNEDGRFTAQLNRASQYISLLIRDSKLSDSATYLC |
| | AVISAGTALIFGKGTTLSVSS (SEQ ID NO: 4) |
| β | DAGVTQSPTHLIKTRGQQVTLRCSPKSGHDTVSWYQQALGQGPQ |
| chain | FIFQYYEEEERQRGNFPDRFSGHQFPNYSSELNVNALLLGDSAL |
| | YLCASSLGWRGGLYTEAFFGQGTRLTVV (SEQ ID NO: 9) |

TABLE 2
Coding sequences of the variable
regions of CRTE7A2

| | |
|---|---|
| α | CGGAAGGAGGTGGAGCAGGATCCTGGACCCTTCAATGTTCCAGAGGGAGCC |
| chain | ACTGTCGCTTTCAACTGTACTTACAGCAACAGTGCTTCTCAGTCTTTCTTCTG |
| | GTACAGACAGGATTGCAGGAAAGAACCTAAGTTGCTGATGTCCGTATACTC |
| | CAGTGGTAACGAAGATGGAAGGTTTACAGCACAGCTCAATAGAGCCAGCCA |
| | GTATATTTCCCTGCTCATCAGAGACTCCAAGCTCAGTGATTCAGCCACCTAC |
| | CTCTGTGCGGTGATCTCGGCAGGAACTGCTCTGATCTTTGGGAAGGGAACC |
| | ACCTTATCAGTGAGTTCC (SEQ ID NO: 5) |
| β | GACGCTGGAGTCACCCAAAGTCCCACACACCTGATCAAAACGAGAGGACA |
| chain | GCAAGTGACTCTGAGATGCTCTCCTAAGTCTGGGCATGACACTGTGTCCTGG |
| | TACCAACAGGCCCTGGGTCAGGGGCCCCAGTTTATCTTTCAGTATTATGAGG |
| | AGGAAGAGAGACAGAGAGGCAACTTCCCTGATCGATTCTCAGGTCACCAGT |
| | TCCCTAACTATAGCTCTGAGCTGAATGTGAACGCCTTGTTGCTGGGGGACTC |
| | GGCCCTCTATCTCTGTGCCAGCAGCTTGGGGTGGCGGGGAGGTCTTTACACT |
| | GAAGCTTTCTTTGGACAAGGCACCAGACTCACAGTTGTA (SEQ ID NO: 10) |

TABLE 3
CDR sequences of a chain and B chain variable
regions of CRTE7A2

| | α chain | β chain |
|---|---|---|
| CDR1 | NSASQS (SEQ ID NO: 1) | SGHDT (SEQ ID NO: 6) |
| CDR2 | VYSSGN (SEQ ID NO: 2) | YYEEEE (SEQ ID NO: 7) |
| CDR3 | AVISAGTALI | ASSLGWRGGLYTEAF |
| | (SEQ ID NO: 3) | (SEQ ID NO: 8) |

TABLE 4
Sequences of the a chain and B chain constant regions
of the recombinant TCR
α chain

| | |
|---|---|
| Amino acid | NIQNPDPAVYQLRDSKSSDKSVCLFTDFDSQTNVSQSKDSDVYITDKT |
| sequence of | VLDMRSMDFKSNSAVAWSNKSDFACANAFNNSIIPEDTFFPSPESSCDV |
| wild-type | KLVEKSFETDTNLNFQNLSVIGFRILLLKVAGFNLLMTLRLWSS (SEQ |
| (WT) human | ID NO: 14) |
| TRAC | |
| Amino acid | NIQNPDPAVYQLRDSKSSDKSVCLFTDFDSQTNVSQSKDSDVYITDKC |
| sequence of | VLDMRSMDFKSNSAVAWSNKSDFACANAFNNSIIPEDTFFPSPESSCDV |
| human TRAC | KLVEKSFETDTNLNFQNLSVIGFRILLLKVAGFNLLMTLRLWSS (SEQ |
| variant 1 | ID NO: 15) |
| (T48C) | |
| Amino acid | NIQNPDPAVYQLRDSKSSDKSVCLFTDFDSQTNVSQSKDSDVYITDKC |
| sequence of | VLDMRSMDFKSNSAVAWSNKSDFACANAFNNSIIPEDTFFPSPESSCDV |
| human TRAC | KLVEKSFETDTNLNFQNLLVIVLRILLLKVAGFNLLMTLRLWSS (SEQ |
| variant 2 | ID NO: 16) |
| (T48C + LVL | |
| mutation) | |
| Amino acid | NIQNPEPAVYQLKDPRSQDSTLCLFTDFDSQINVPKTMESGTFITDKTV |
| sequence of | LDMKAMDSKSNGAIAWSNQTSFTCQDIFKETNATYPSSDVPCDATLTE |
| WT murine | KSFETDMNLNFQNLSVMGLRILLLKVAGENLLMTLRLWSS (SEQ ID |
| TRAC | NO: 17) |
| Amino acid | NIQNPEPAVYQLKDPRSQDSTLCLFTDFDSQINVPKTMESGTFITDKCV |
| sequence of | LDMKAMDSKSNGAIAWSNQTSFTCQDIFKETNATYPSSDVPCDATLTE |
| murine TRAC | KSFETDMNLNFQNLSVMGLRILLLKVAGFNLLMTLRLWSS (SEQ ID |
| variant 1 | NO: 18) |
| (T48C) | |
| Amino acid | NIQNPEPAVYQLKDPRSQDSTLCLFTDFDSQINVPKTMESGTFITDKCV |
| sequence of | LDMKAMDSKSNGAIAWSNQTSFTCQDIFKETNATYPSSDVPCDATLTE |
| murine TRAC | KSFETDMNLNFQNLLVIVLRILLLKVAGENLLMTLRLWSS (SEQ ID |
| variant 2 | NO: 19) |
| (T48C + LIV | |
| mutation) | |
| Coding | AATATTCAGAACCCCGAACCAGCCGTATATCAGTTGAAGGACCCAA |
| sequence of | GATCTCAGGATAGTACACTCTGCCTGTTCACCGACTTTGACTCACAA |
| murine TRAC | ATCAACGTCCCGAAGACTATGGAAAGTGGTACGTTCATCACAGATA |
| variant 2 | AGTGCGTTCTGGACATGAAGGCTATGGACTCAAAGAGCAACGGGG |
| (T48C + LIV | CAATTGCTTGGTCCAACCAGACAAGCTTTACCTGTCAGGACATTTTT |
| mutation) | AAGGAGACTAATGCTACTTATCCCTCCAGCGACGTTCCGTGTGATG |
| | CGACTCTTACCGAGAAGTCTTTTGAGACCGATATGAATCTCAACTTC |
| | CAGAATCTGCTGGTGATCGTTCTGCGGATCCTGCTTCTGAAGGTTGC |
| | AGGATTCAATCTTCTTATGACTCTCCGGCTCTGGTCTTCA (SEQ ID |
| | NO: 20) |

β chain

| | |
|---|---|
| Amino acid | EDLNKVFPPEVAVFEPSEAEISHTQKATLVCLATGFFPDHVELSWWVN |
| sequence of | GKEVHSGVSTDPQPLKEQPALNDSRYCLSSRLRVSATFWQNPRNHFRC |
| WT human | QVQFYGLSENDEWTQDRAKPVTQIVSAEAWGRADCGFTSVSYQQGVL |
| TRBC1 | SATILYEILLGKATLYAVLVSALVLMAMVKRKDF (SEQ ID NO: 21) |
| Amino acid | EDLNKVFPPEVAVFEPSEAEISHTQKATLVCLATGFFPDHVELSWWVN |
| sequence of | GKEVHSGVCTDPQPLKEQPALNDSRYCLSSRLRVSATFWQNPRNHFRC |
| human | QVQFYGLSENDEWTQDRAKPVTQIVSAEAWGRADCGFTSVSYQQGVL |
| TRBC1 | SATILYEILLGKATLYAVLVSALVLMAMVKRKDF (SEQ ID NO: 22) |
| variant (S57C) | |
| Amino acid | EDLKNVFPPKVAVFEPSEAEISHTQKATLVCLATGFYPDHVELSWWVN |
| sequence of | GKEVHSGVSTDPQPLKEQPALNDSRYCLSSRLRVSATFWQNPRNHFRC |
| WT human | QVQFYGLSENDEWTQDRAKPVTQIVSAEAWGRADCGFTSESYQQGVLSA |
| TRBC2 | TILYEILLGKATLYAVLVSALVLMAMVKRKDSRG (SEQ ID NO: 23) |
| Amino acid | EDLKNVFPPKVAVFEPSEAEISHTQKATLVCLATGFYPDHVELSWWVN |
| sequence of | GKEVHSGVCTDPQPLKEQPALNDSRYCLSSRLRVSATFWQNPRNHFRC |
| human | QVQFYGLSENDEWTQDRAKPVTQIVSAEAWGRADCGFTSESYQQGVLSA |
| TRBC2 | TILYEILLGKATLYAVLVSALVLMAMVKRKDSRG (SEQ ID NO: 24) |
| variant (S57C) | |
| Amino acid | EDLRNVTPPKVSLFEPSKAEIANKQKATLVCLARGFFPDHVELSWWVN |
| sequence of | GKEVHSGVSTDPQAYKESNYSYCLSSRLRVSATFWHNPRNHFRCQVQ |
| WT murine | FHGLSEEDKWPEGSPKPVTQNISAEAWGRADCGITSASYQQGVLSATIL |
| TRBC1 | YEILLGKATLYAVLVSTLVVMAMVKRKNS (SEQ ID NO: 25) |
| Amino acid | EDLRNVTPPKVSLFEPSKAEIANKQKATLVCLARGFFPDHVELSWWVN |
| sequence of | GKEVHSGVCTDPQAYKESNYSYCLSSRLRVSATFWHNPRNHFRCQVQ |
| murine | FHGLSEEDKWPEGSPKPVTQNISAEAWGRADCGITSASYQQGVLSATIL |
| TRBC1 | YEILLGKATLYAVLVSTLVVMAMVKRKNS (SEQ ID NO: 26) |
| variant (S57C) | |
| Coding | GAGGACCTGCGCAACGTCACCCCACCAAAGGTCAGTTTGTTTGAGC |
| sequence of | CATCAAAGGCGGAGATCGCCAACAAACAGAAAGCTACCCTCGTGT |
| murine | GCTTGGCCCGGGGCTTCTTCCCAGACCACGTAGAACTTTCCTGGTGG |
| TRBC1 | GTCAATGGAAAGGAGGTTCATTCCGGAGTGTGCACTGATCCCCAAG |
| variant (S57C) | CGTACAAGGAATCCAACTATAGCTACTGTCTCTCATCTCGGCTCCGG |
| | GTGAGTGCGACATTCTGGCATAATCCTCGGAACCACTTTCGATGCC |
| | AAGTGCAGTTTCATGGGTTGAGCGAGGAAGACAAGTGGCCCGAGG |
| | GCAGTCCTAAACCAGTCACTCAAAACATAAGCGCCGAGGCATGGG |
| | GTAGAGCCGATTGTGGGATTACTAGCGCTTCATACCAACAAGGGGT |
| | ATTGAGCGCTACAATTCTTTACGAAATTCTCCTCGGCAAGGCGACG |
| | CTCTACGCCGTACTGGTGTCTACTCTCGTGGTTATGGCAATGGTGAA |
| | ACGGAAAAACAGC (SEQ ID NO: 31) |
| Amino acid | EDLRNVTPPKVSLFEPSKAEIANKQKATLVCLARGFFPDHVELSWWVN |
| sequence of | GKEVHSGVSTDPQAYKESNYSYCLSSRLRVSATFWHNPRNHFRCQVQ |
| WT murine | FHGLSEEDKWPEGSPKPVTQNISAEAWGRADCGITSASYQQGVLSATIL |
| TRBC1′ | YEILLGKATLYAVLVSTLVVMAMVRNR (SEQ ID NO: 27) |
| Amino acid | EDLRNVTPPKVSLFEPSKAEIANKQKATLVCLARGFFPDHVELSWWVN |
| sequence of | GKEVHSGVCTDPQAYKESNYSYCLSSRLRVSATFWHNPRNHFRCQVQ |
| murine | FHGLSEEDKWPEGSPKPVTQNISAEAWGRADCGITSASYQQGVLSATIL |
| TRBC1′ | YEILLGKATLYAVLVSTLVVMAMVRNR (SEQ ID NO: 28) |
| variant (S57C) | |
| Amino acid | EDLRNVTPPKVSLFEPSKAEIANKQKATLVCLARGFFPDHVELSWWVN |
| sequence of | GKEVHSGVSTDPQAYKESNYSYCLSSRLRVSATFWHNPRNHFRCQVQ |
| WT murine | FHGLSEEDKWPEGSPKPVTQNISAEAWGRADCGITSASYHQGVLSATIL |
| TRBC2 | YEILLGKATLYAVLVSGLVLMAMVKKKNS (SEQ ID NO: 29) |
| Amino acid | EDLRNVTPPKVSLFEPSKAEIANKQKATLVCLARGFFPDHVELSWWVN |
| sequence of | GKEVHSGVCTDPQAYKESNYSYCLSSRLRVSATFWHNPRNHFRCQVQ |
| murine | FHGLSEEDKWPEGSPKPVTQNISAEAWGRADCGITSASYHQGVLSATIL |
| TRBC2 | YEILLGKATLYAVLVSGLVLMAMVKKKNS (SEQ ID NO: 30) |
| variant (S57C) | |

TABLE 5
Amino acid sequences of the a chain and B chain of the
recombinant TCR

| | |
|---|---|
| α chain | MISLRVLLVILWLQLSWVWSQRKEVEQDPGPFNVPEGATVAFNCTYSNS |
| | ASQSFFWYRQDCRKEPKLLMSVYSSGNEDGRFTAQLNRASQYISLLIRDS |
| | KLSDSATYLCAVISAGTALIFGKGTTLSVSSNIQNPDPAVYQLRDSKSSDK |
| | SVCLFTDFDSQTNVSQSKDSDVYITDKTVLDMRSMDFKSNSAVAWSNKS |
| | DFACANAFNNSIIPEDTFFPSPESSCDVKLVEKSFETDTNLNFQNLSVIGFRI |
| | LLLKVAGFNLLMTLRLWSS (SEQ ID NO: 32) |
| | MISLRVLLVILWLQLSWVWSQRKEVEQDPGPFNVPEGATVAFNCTYSNS |
| | ASQSFFWYRQDCRKEPKLLMSVYSSGNEDGRFTAQLNRASQYISLLIRDS |
| | KLSDSATYLCAVISAGTALIFGKGTTLSVSSNIQNPDPAVYQLRDSKSSDK |
| | SVCLFTDFDSQTNVSQSKDSDVYITDKCVLDMRSMDFKSNSAVAWSNKS |
| | DFACANAFNNSIIPEDTFFPSPESSCDVKLVEKSFETDTNLNFQNLSVIGFRI |
| | LLLKVAGFNLLMTLRLWSS (SEQ ID NO: 33) |
| | MISLRVLLVILWLQLSWVWSQRKEVEQDPGPFNVPEGATVAFNCTYSNS |
| | ASQSFFWYRQDCRKEPKLLMSVYSSGNEDGRFTAQLNRASQYISLLIRDS |
| | KLSDSATYLCAVISAGTALIFGKGTTLSVSSNIQNPDPAVYQLRDSKSSDK |
| | SVCLFTDFDSQTNVSQSKDSDVYITDKCVLDMRSMDFKSNSAVAWSNKS |
| | DFACANAFNNSIIPEDTFFPSPESSCDVKLVEKSFETDTNLNFQNLLVIVLRI |
| | LLLKVAGFNLLMTLRLWSS (SEQ ID NO: 34) |
| | MISLRVLLVILWLQLSWVWSQRKEVEQDPGPFNVPEGATVAFNCTYSNS |
| | ASQSFFWYRQDCRKEPKLLMSVYSSGNEDGRFTAQLNRASQYISLLIRDS |
| | KLSDSATYLCAVISAGTALIFGKGTTLSVSSNIQNPEPAVYQLKDPRSQDST |
| | LCLFTDFDSQINVPKTMESGTFITDKTVLDMKAMDSKSNGAIAWSNQTSF |
| | TCQDIFKETNATYPSSDVPCDATLTEKSFETDMNLNFQNLSVMGLRILLLK |
| | VAGFNLLMTLRLWSS (SEQ ID NO: 35) |
| | MISLRVLLVILWLQLSWVWSQRKEVEQDPGPFNVPEGATVAFNCTYSNS |
| | ASQSFFWYRQDCRKEPKLLMSVYSSGNEDGRFTAQLNRASQYISLLIRDS |
| | KLSDSATYLCAVISAGTALIFGKGTTLSVSSNIQNPEPAVYQLKDPRSQDST |
| | LCLFTDFDSQINVPKTMESGTFITDKCVLDMKAMDSKSNGAIAWSNQTSF |
| | TCQDIFKETNATYPSSDVPCDATLTEKSFETDMNLNFQNLSVMGLRILLLK |
| | VAGFNLLMTLRLWSS (SEQ ID NO: 36) |
| | MISLRVLLVILWLQLSWVWSQRKEVEQDPGPFNVPEGATVAFNCTYSNS |
| | ASQSFFWYRQDCRKEPKLLMSVYSSGNEDGRFTAQLNRASQYISLLIRDS |
| | KLSDSATYLCAVISAGTALIFGKGTTLSVSSNIQNPEPAVYQLKDPRSQDST |
| | LCLFTDFDSQINVPKTMESGTFITDKCVLDMKAMDSKSNGAIAWSNQTSF |
| | TCQDIFKETNATYPSSDVPCDATLTEKSFETDMNLNFQNLLVIVLRILLLK |
| | VAGFNLLMTLRLWSS (SEQ ID NO: 37) |
| β chain | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLNKVFPPEVAVF |
| | EPSEAEISHTQKATLVCLATGFFPDHVELSWWVNGKEVHSGVSTDPQPLK |
| | EQPALNDSRYCLSSRLRVSATFWQNPRNHFRCQVQFYGLSENDEWTQDR |
| | AKPVTQIVSAEAWGRADCGFTSVSYQQGVLSATILYEILLGKATLYAVLV |
| | SALVLMAMVKRKDF (SEQ ID NO: 38) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLNKVFPPEVAVF |
| | EPSEAEISHTQKATLVCLATGFFPDHVELSWWVNGKEVHSGVCTDPQPLK |
| | EQPALNDSRYCLSSRLRVSATFWQNPRNHFRCQVQFYGLSENDEWTQDR |
| | AKPVTQIVSAEAWGRADCGFTSVSYQQGVLSATILYEILLGKATLYAVLV |
| | SALVLMAMVKRKDF (SEQ ID NO: 39) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLKNVFPPKVAV |
| | FEPSEAEISHTQKATLVCLATGFYPDHVELSWWVNGKEVHSGVSTDPQPL |
| | KEQPALNDSRYCLSSRLRVSATFWQNPRNHFRCQVQFYGLSENDEWTQD |
| | RAKPVTQIVSAEAWGRADCGFTSESYQQGVLSATILYEILLGKATLYAVL |
| | VSALVLMAMVKRKDSRG (SEQ ID NO: 40) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLKNVFPPKVAV |
| | FEPSEAEISHTQKATLVCLATGFYPDHVELSWWVNGKEVHSGVCTDPQPL |
| | KEQPALNDSRYCLSSRLRVSATFWQNPRNHFRCQVQFYGLSENDEWTQD |
| | RAKPVTQIVSAEAWGRADCGFTSESYQQGVLSATILYEILLGKATLYAVL |
| | VSALVLMAMVKRKDSRG (SEQ ID NO: 41) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLRNVTPPKVSLF |
| | EPSKAEIANKQKATLVCLARGFFPDHVELSWWVNGKEVHSGVSTDPQAY |
| | KESNYSYCLSSRLRVSATFWHNPRNHFRCQVQFHGLSEEDKWPEGSPKPV |
| | TQNISAEAWGRADCGITSASYQQGVLSATILYEILLGKATLYAVLVSTLVV |
| | MAMVKRKNS (SEQ ID NO: 42) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLRNVTPPKVSLF |
| | EPSKAEIANKQKATLVCLARGFFPDHVELSWWVNGKEVHSGVCTDPQAY |
| | KESNYSYCLSSRLRVSATFWHNPRNHFRCQVQFHGLSEEDKWPEGSPKPV |
| | TQNISAEAWGRADCGITSASYQQGVLSATILYEILLGKATLYAVLVSTLVV |
| | MAMVKRKNS (SEQ ID NO: 43) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLRNVTPPKVSLF |
| | EPSKAEIANKQKATLVCLARGFFPDHVELSWWVNGKEVHSGVSTDPQAY |
| | KESNYSYCLSSRLRVSATFWHNPRNHFRCQVQFHGLSEEDKWPEGSPKPV |
| | TQNISAEAWGRADCGITSASYQQGVLSATILYEILLGKATLYAVLVSTLVV |
| | MAMVRNR (SEQ ID NO: 44) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLRNVTPPKVSLF |
| | EPSKAEIANKQKATLVCLARGFFPDHVELSWWVNGKEVHSGVCTDPQAY |
| | KESNYSYCLSSRLRVSATFWHNPRNHFRCQVQFHGLSEEDKWPEGSPKPV |
| | TQNISAEAWGRADCGITSASYQQGVLSATILYEILLGKATLYAVLVSTLVV |
| | MAMVRNR (SEQ ID NO: 45) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLRNVTPPKVSLF |
| | EPSKAEIANKQKATLVCLARGFFPDHVELSWWVNGKEVHSGVSTDPQAY |
| | KESNYSYCLSSRLRVSATFWHNPRNHFRCQVQFHGLSEEDKWPEGSPKPV |
| | TQNISAEAWGRADCGITSASYHQGVLSATILYEILLGKATLYAVLVSGLVL |
| | MAMVKKKNS (SEQ ID NO: 46) |
| | MGPGLLCWALLCLLGAGLVDAGVTQSPTHLIKTRGQQVTLRCSPKSGHD |
| | TVSWYQQALGQGPQFIFQYYEEEERQRGNFPDRFSGHQFPNYSSELNVNA |
| | LLLGDSALYLCASSLGWRGGLYTEAFFGQGTRLTVVEDLRNVTPPKVSLF |
| | EPSKAEIANKQKATLVCLARGFFPDHVELSWWVNGKEVHSGVCTDPQAY |
| | KESNYSYCLSSRLRVSATFWHNPRNHFRCQVQFHGLSEEDKWPEGSPKPV |
| | TQNISAEAWGRADCGITSASYHQGVLSATILYEILLGKATLYAVLVSGLVL |
| | MAMVKKKNS (SEQ ID NO: 47) |

The α chain and β chain variable regions of CRTE7A2 were fused to the murine constant regions (SEQ ID NO:19 (α chain) and SEQ ID NO:26 (β chain)) and constructed in tandem in β-T2A-α order into the lentiviral vector for subsequent use.

### EXAMPLE 2

### Binding Affinity of CRTE7A2 Against Target Antigenic Peptide

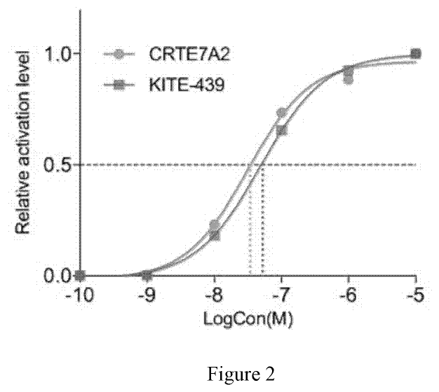

To confirm the affinity of CRTE7A2 against the target antigenic peptide (YMLDLQPET, SEQ ID NO:11), KITE-439, a TCR targeting HPV16 E7 developed by Kite Pharma, was used as a control. CRTE7A2 and KITE-439 were introduced into Jurkat cells carrying the NFAT-GFP reporter gene (Jurkat-NFAT-GFP, with the endogenous TCR knocked out) using lentivirus. The Jurkat cells were co-incubated with T2 cells loaded with various concentrations of the target antigenic peptide, and the activation level of the reporter gene in the Jurkat cells was measured. The results show that CRTE7A2 TCR-T cells exhibit strong reactivity against the target antigenic peptide that is superior to that of KITE-439 TCR-T cells, with the EC50 of CRTE7A2 binding to the target antigen being 3.10*10⁻⁸ M and the EC50 of KITE-439 binding to the target antigen being 4.85*10⁻⁸ M (FIG. 2). The above results indicate that CRTE7A2 has high binding affinity against the target antigen peptide.

### EXAMPLE 3

### Cell Membrane Expression Stability of CRTE7A2

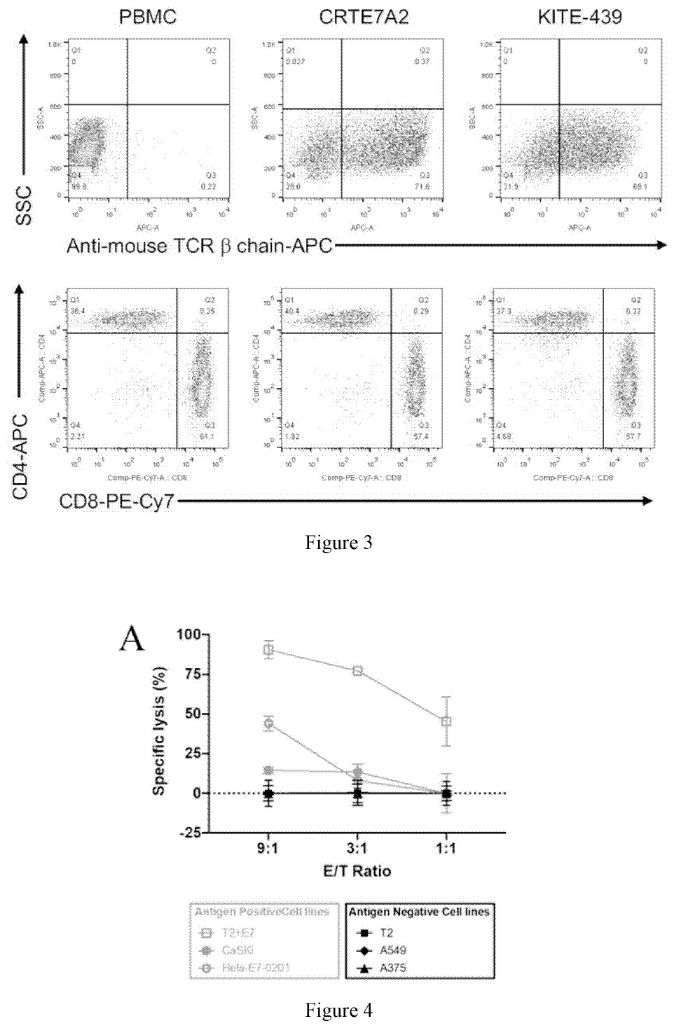

PBMCs were transduced with equal amounts of KITE-439 and CRTE7A2 lentivirus (MOI20) and then amplified and cultured until day 9. Successful TCR transduction was confirmed using human CD3-FITC antibody. The expression rate of TCR-T was detected by staining using anti-mouse TCR β-APC antibody; and the ratio of CD4/CD8 was measured by staining using human CD4-APC and CD8-PE-Cy7 antibodies. The results of flow cytometry show that the ratio of CD4/CD8 and TCR positive rate of each cell after the transduction of both TCRs are substantially the same under the same transduction and culture conditions (FIG. 3). The expression intensity of CRTE7A2 is significantly better than that of KITE-439, indicating that CRTE7A2 TCR has better cell membrane expression stability and suggesting that CRTE7A2 potentially has better antigen reactivity and anti-tumor activity.

### EXAMPLE 4

### Specific Killing Activity of CRTE7A2 TCR-T Cells Against Antigen-Positive Tumor Cells

T cells expressing CRTE7A2 or KITE-439 TCR were used as effector cells, and PBMCs not transduced with TCRs amplified and cultured in parallel were used as a control for effector cells. T2 cells loaded with 10⁻⁶ M of HPV16 E7₁₁₋₁₉ polypeptide (HLA-A*02:01 positive), Caski cells (HPV16 E7 positive, HLA-A*02:01 positive) and Hela-E7-0201 cells (over-expressing HPV16 E7 and HLA-A*02:01) were used as HLA-antigen peptide-matched positive target cells. T2 cells not loaded with HPV16 E7₁₁₋₁₉ polypeptide, A375 cells (HPV16 E7 negative, HLA-A*02:01 positive) and A549 cells (HPV16 E7 negative, HLA-A*02:01 negative) were used as HLA-antigen peptide-unmatched negative target cells. All target cells stably expressed the luciferase gene. Effector cells were incubated with different target cells at the effector cell to target cell (E: T) ratios of 9:1, 3:1 and 1:1, respectively, for 16 hr, and luciferase substrate was added to detect the surviving target cells. The ratio of the killed target cells was calculated based on the remaining target cells.

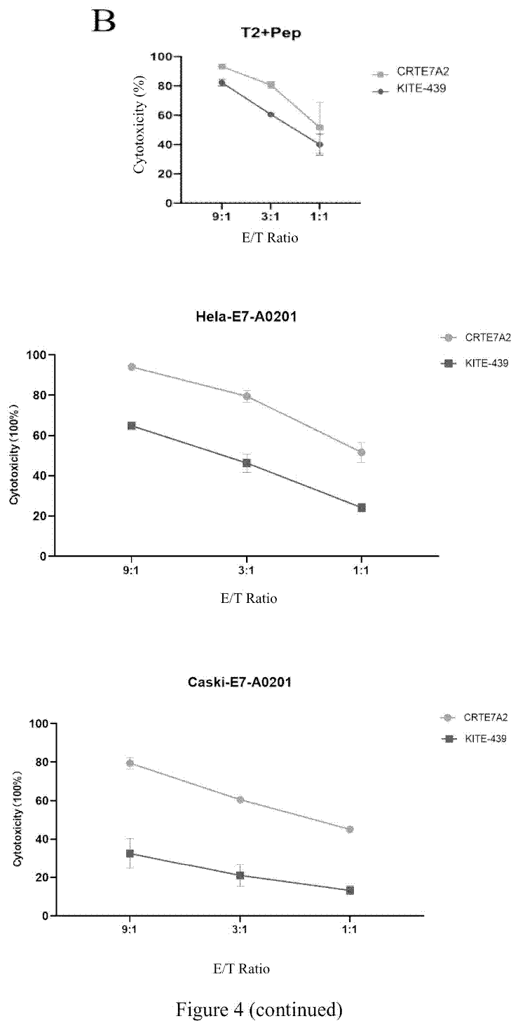

The results show that CRTE7A2 TCR-T cells has a significant killing effect against positive target cells and no killing effect against negative target cells (FIG. 4A), indicating that CRTE7A2 TCR-T cells have the ability to specifically kill the HLA-antigen peptide-matched target cells. Comparison with the results of KITE-439 show that CRTE7A2 TCR-T cells exhibit significantly superior killing activity against target cells than KITE-439 (FIG. 4B). These results indicate a significantly more efficient specific killing effect of CRTE7A2 TCR-T cells against antigen-positive tumor cells.

### EXAMPLE 5

### Specific Response of CRTE7A2 TCR-T Cells Against Antigen-Positive Target Cells

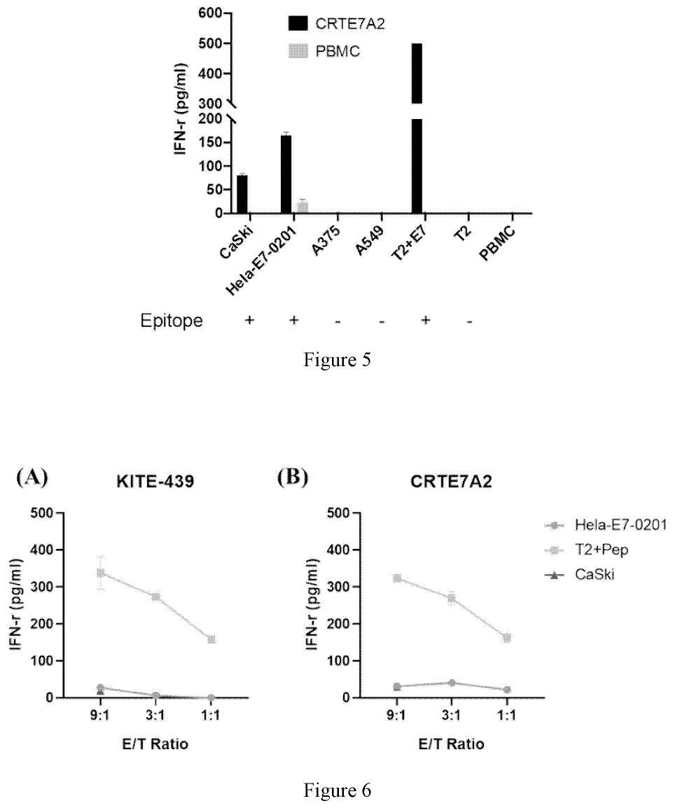

T cells expressing CRTE7A2 TCR were used as effector cells, and PBMCs not transduced with TCRs amplified and cultured in parallel were used as a control for effector cells. T2 cells loaded with 10⁻⁶ M of HPV16 E7₁₁₋₁₉ polypeptide (HLA-A*02:01 positive), Caski cells (HPV16 E7 positive, HLA-A*02:01 positive) and Hela-E7-0201 cells (over-expressing HPV16 E7 and HLA-A*02:01) were used as HLA-antigen peptide-matched positive target cells. T2 cells not loaded with HPV16 E7₁₁₋₁₉ polypeptide, A375 cells (HPV16 E7 negative, HLA-A*02:01 positive) and A549 cells (HPV16 E7 negative, HLA-A*02:01 negative) were used as HLA-antigen peptide-unmatched negative target cells. The number of CRTE7A2 TCR-T cells was 10⁵. CRTE7A2 TCR-T cells were incubated with various target cells at the E: T ratio of 1:1 for 24 hr, and the secretion of IFN-γ in supernatant was detected using the IFN-γ ELISA kit (Thermo, Cat #88-7316-76). The results are as shown in FIG. 5.

The results show that CRTE7A2 TCR-T cells produce IFN-γ secretion against HLA-antigen peptide-matched positive target cells and no IFN-γ secretion against HLA-antigen peptide-unmatched negative target cells, indicating the good specific killing effect of CRTE7A2 TCR-T cells.

TCR-T cells expressing CRTE7A2 or KITE-439 were used as effector cells. T2 cells loaded with 10-6 M of HPV16 E7₁₁₋₁₉ polypeptide (HLA-A*02:01 positive), Caski cells (HPV16 E7positive, HLA-A*02:01 positive) and Hela-E7-0201 cells (over-expressing HPV16 E7 and HLA-A*02:01) were used as HLA-antigen peptide-matched positive target cells. The number of CRTE7A2 TCR-T cells was 105. CRTE7A2 TCR-T cells were incubated with various target cells at the E: T ratios of 9:1, 3:1 and 1:1, respectively, for 24 hr, and the secretion of IFN-γ in supernatant was detected. The results are as shown in FIG. 6. The results show that similar to KITE-439 TCR-T cells, CRTE7A2 TCR-T cells are able to secrete high levels of IFN-γ under the stimulation of antigen-positive target cells.

### EXAMPLE 6

### Specific IFN-γ Secretion by CRTE7A2 TCR-T Cells Against Antigen-Positive Target Cells

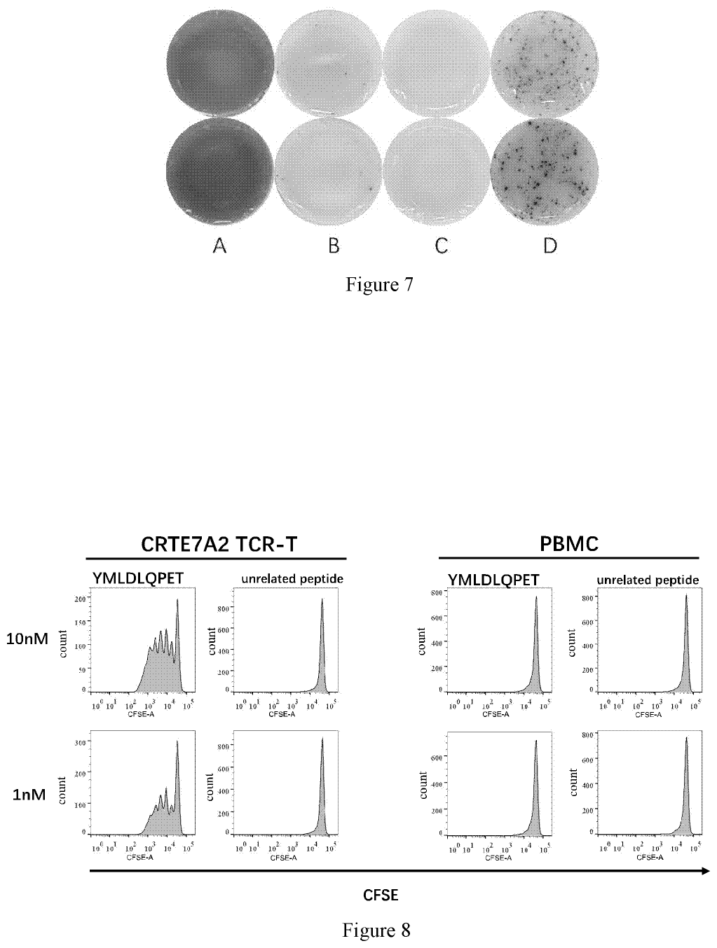

10⁵ of CRTE7A2 TCR-T cells were added to ELISpot detection wells A, B, C, and D that are capable of capturing IFN-γ secreted by cells. To different wells, added with: (A) 25 ng/ml phytohemagglutinin (PHA; T-cell cytokinin) as a positive control; (B) CRTE7A2 TCR-T cells as a self-control; (C) culture medium for culture of CRTE7A2 as a negative control; and (D) HLA-A*02:01-positive T2 cells loaded with 10 nM HPV16 E7₁₁₋₁₉ polypeptide as experimental wells. ELISpot staining was performed after incubation for 16-24 hr, and the results are as shown in FIG. 7.

The results show that CRTE7A2 in well A exhibits a brown positive response to PHA stimulation; wells B and C served as negative controls exhibit no obvious brown spots; and CRTE7A2 in well D exhibits obvious brown spots under the stimulation of 10 nM HPV16 E7₁₁₋₁₉ polypeptide. This indicates that CRTE7A2 produces specific IFN-γ secretion under the stimulation of HPV16 E7₁₁₋₁₉ polypeptide presented by HLA-A*02:01-positive T2 cells.

### EXAMPLE 7

### CRTE7A2 Efficiently Mediates Antigen-Specific T Cell Proliferation

CRTE7A2 TCR-T cells and PBMCs not transduced with TCRs were rested in IL-2 free, lymphocyte serum-free medium X VIVO-15 (Lonza, Cat. BE02-060F) for 24 hr, respectively, then stained and labeled with CFSE (C34554, Invitrogen), and were co-cultured with T2 cells loaded with 10 nM or 1 nM HPV16 E7₁₁₋₁₉ polypeptide for 5 days. Then the mixed cells were stained for mouse TCRB-antigen presenting cells (APC) and human CD8α-PE Cy7. CD8 and mouse TCRB-positive cells were selected for analysis of cell proliferation. T2 cells loaded with irrelevant polypeptide were used as a control for the polypeptide and PBMCs not transduced with TCRs were used as a negative control. The results are as shown in FIG. 8.

The results show that the HPV16 E711-19 polypeptide is able to specifically stimulate the proliferation of CRTE7A2 TCR-T cells, which further confirms the antigen-specific activation of CRTE7A2 TCR-T cells.

### EXAMPLE 8

### In Vivo Anti-Tumor Activity of CRTE7A2

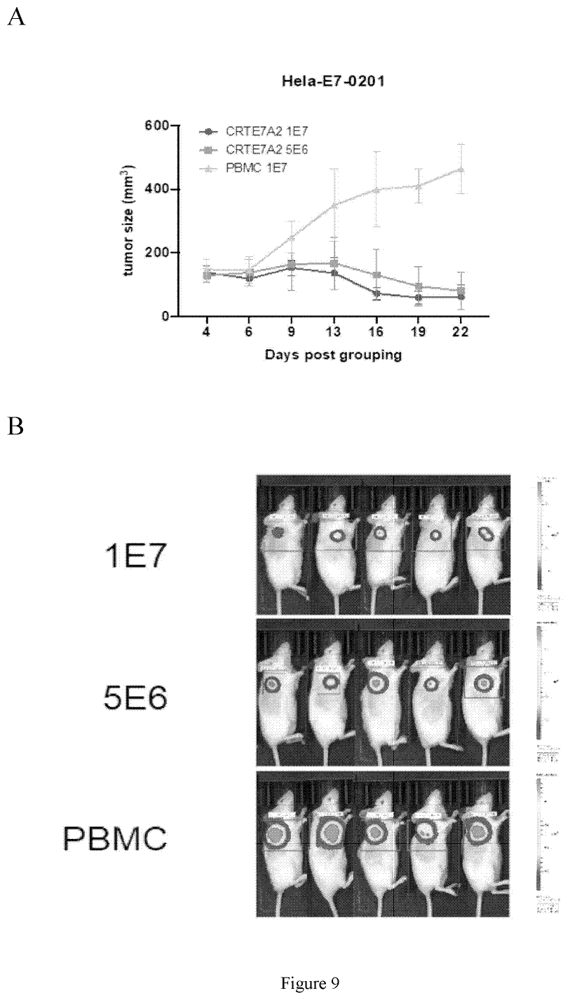

A tumorigenic model (cell line-derived xenograft (CDX) model) was established in a B2M-knockout NDG immunodeficient mouse using a cervical cancer cell line Hela-E7-A0201, followed by tail vein reinfusion of 5×10⁶ and 10⁷ of CRTE7A2 TCR-T cells and reinfusion of 10⁷ PBMCs as a control. The growth of subcutaneous transplanted Hela tumor was monitored, and the results are as shown in FIG. 9A. The results demonstrate that CRTE7A2 TCR-T cells has dose-dependent tumor inhibition activity. 5×10⁶ and 10⁷ of CRTE7A2 TCR-T cells may significantly inhibit the growth of tumor cells, and 10⁷ cells have a better tumor inhibition effect than that of 5×10⁶ cells. The growth of subcutaneous transplanted Hela tumor monitored after 21 days of TCR-T cell reinfusion using luciferase live imaging system show that reinfusion of 5×10⁶ and 10⁷ of CRTE7A2 TCR-T cells may significantly inhibit the growth of tumor cells, and 10⁷ cells have a better tumor inhibition effect than that of 5×10⁶ cells (FIG. 9B).

These results suggest that CRTE7A2 TCR-T cells have a significant in vivo anti-tumor activity.

The following embodiments are herewith listed for the present application.

1. An antigen binding protein comprising a T cell receptor (TCR) alpha chain variable region and a TCR beta chain variable region, wherein the TCR alpha chain variable region comprises a CDR3 having the amino acid sequence: AVISAGTALI (SEQ ID NO:3), or a functional variant thereof formed by insertion, deletion or substitution of one or more amino acids,

wherein the antigen binding protein binds to an epitope comprising the amino acid sequence of YMLDLQPET (SEQ ID NO:11) or a complex of the epitope with an MHC molecule.

2. An antigen binding protein comprising a T cell receptor (TCR) alpha chain variable region and a TCR beta chain variable region, wherein the TCR beta chain variable region comprises a CDR3 having the amino acid sequence: ASSLGWRGGLYTEAF (SEQ ID NO:8), or a functional variant thereof formed by insertion, deletion or substitution of one or more amino acids,

wherein the antigen binding protein binds to an epitope comprising the amino acid sequence of YMLDLQPET (SEQ ID NO: 11) or a complex of the epitope with an MHC molecule.

3. An antigen binding protein comprising a T cell receptor (TCR) alpha chain variable region and a TCR beta chain variable region, wherein the TCR alpha chain variable region comprises a CDR3 having the amino acid sequence: AVISAGTALI (SEQ ID NO:3), or a functional variant thereof formed by insertion, deletion or substitution of one or more amino acids; and wherein the TCR beta chain variable region comprises a CDR3 having the amino acid sequence: ASSLGWRGGLYTEAF (SEQ ID NO:8), or a functional variant thereof formed by insertion, deletion or substitution of one or more amino acids,

wherein the antigen binding protein binds to an epitope comprising the amino acid sequence of YMLDLQPET (SEQ ID NO: 11) or a complex of the epitope with an MHC molecule.

4. The antigen binding protein according to any one of embodiments 1-3, wherein the MHC molecule is HLA-A*02 type, such as HLA-A*02:01 type, HLA-A*02:03 type, HLA-A*02:05 type, HLA-A*02:06 type, HLA-A*02:07 type, HLA-A*02:10 type, or HLA-A*02:11 type.

5. The antigen binding protein according to any one of embodiments 1-4, wherein the TCR alpha chain variable region comprises a CDR3 having the amino acid sequence as shown in SEQ ID NO: 3, and/or the TCR beta chain variable region comprises a CDR3 having the amino acid sequence as shown in SEQ ID NO:8.

6. The antigen binding protein according to any one of embodiments 1-5, wherein the TCR alpha chain variable region comprises CDR1, CDR2 and CDR3 having the amino acid sequences as shown in SEQ ID NO:1, SEQ ID NO:2 and SEQ ID NO:3, respectively, or a functional variant thereof formed by insertion, deletion or substitution of one or more amino acids; and/or the TCR beta chain variable region comprises beta chain CDR1, CDR2 and CDR3 having the amino acid sequences as shown in SEQ ID NO:6, SEQ ID NO:7 and SEQ ID NO:8, respectively, or a functional variant thereof formed by insertion, deletion or substitution of one or more amino acids.

7. An antigen binding protein comprising a T cell receptor (TCR) alpha chain variable region and a TCR beta chain variable region, wherein the TCR alpha chain variable region comprises CDR1, CDR2, and CDR3 having the amino acid sequences as shown in SEQ ID NO: 1, SEQ ID NO:2, and SEQ ID NO:3, respectively; and/or the TCR beta chain variable region comprises beta chain CDR1, CDR2, and CDR3 having the amino acid sequences as shown in SEQ ID NO:6, SEQ ID NO:7 and SEQ ID NO:8, respectively.

8. The antigen binding protein according to any one of embodiments 1-7, wherein the TCR alpha chain variable region comprises an amino acid sequence having at least 80%, at least 85%, at least 90%, at least 95% or 100% sequence identity to SEQ ID NO:4, and/or the TCR beta chain variable region comprises an amino acid sequence having at least 80%, at least 85%, at least 90%, at least 95% or 100% sequence identity to SEQ ID NO:9.

9. The antigen binding protein according to embodiment 8, wherein the TCR alpha chain variable region comprises the amino acid sequence as shown in SEQ ID NO:4, and/or the TCR beta chain variable region comprises the amino acid sequence as shown in SEQ ID NO:9.

10. The antigen binding protein according to any one of embodiments 1-9, wherein the TCR alpha chain variable region is comprised on a first polypeptide and the TCR beta chain variable region is comprised on a different second polypeptide.

11. The antigen binding protein according to any one of embodiments 1-9, wherein the TCR alpha chain variable region and the TCR beta chain variable region are comprised on a single polypeptide.

12. The antigen binding protein according to any one of embodiments 1-11, wherein the antigen binding protein is soluble or membrane-bound.

13. The antigen binding protein according to any one of embodiments 1-12, wherein the antigen binding protein is selected from a TCR, a chimeric antigen receptor (CAR), a Fc polypeptide, or an antigen-binding fragment thereof.

14. The antigen binding protein according to any one of embodiments 1-13, wherein the antigen binding protein is a TCR or an antigen-binding fragment thereof, and wherein the antigen binding protein further comprises a TCR constant region or a fragment thereof.

15. The antigen binding protein according to embodiment 14, wherein the TCR constant region is a murine constant region or a human constant region.

16. The antigen binding protein according to embodiment 14 or 15, wherein the TCR constant region comprises a TCR alpha chain constant region and/or a TCR beta chain constant region;

preferably, the TCR alpha chain constant region and/or the TCR beta chain constant region comprises at least one cysteine mutation relative to a wild-type sequence to form a disulfide bond between the TCR alpha chain and the TCR beta chain.

17. The antigen binding protein according to embodiment 16, wherein the TCR alpha chain constant region comprises an amino acid sequence having at least 80%, at least 85%, at least 90%, at least 95%, or 100% sequence identity to any one of SEQ ID NOs: 14-19, and/or the TCR beta chain constant region comprises an amino acid sequence having at least 80%, at least 85%, at least 90%, at least 95%, or 100% sequence identity to any one of SEQ ID NOs: 21-30.

18. The antigen binding protein according to embodiment 17, wherein the TCR alpha chain constant region comprises the amino acid sequence as shown in SEQ ID NO: 19 and the TCR beta chain constant region comprises the amino acid sequence as shown in SEQ ID NO:26.

19. The antigen binding protein according to embodiment 14, wherein the fragment of the TCR constant region is an extracellular segment of the TCR constant region.

20. The antigen binding protein according to any one of embodiments 14-19, wherein the antigen binding protein further comprises a transmembrane region and/or a cytoplasmic region.

21. The antigen binding protein according to embodiment 14, wherein the antigen binding protein comprises a TCR alpha chain comprising an amino acid sequence having at least 80%, at least 85%, at least 90%, at least 95% or 100% sequence identity to any one selected from SEQ ID NOs: 32-37; and/or a TCR beta chain comprising an amino acid sequence having at least 80%, at least 85%, at least 90%, at least 95% or 100% sequence identity to any one selected from SEQ ID NOs: 38-47.

22. The antigen binding protein according to any one of embodiments 1-21, wherein the antigen binding protein further comprises an intracellular signaling region.

23. The antigen binding protein according to any one of embodiments 1-22, wherein the antigen binding protein further comprises one or more antigen-binding region(s) that bind(s) to other antigens or epitopes.

24. The antigen binding protein according to any one of embodiments 1-23, wherein the antigen binding protein is isolated or purified.

25. A nucleic acid encoding the antigen binding protein according to any one of embodiments 1-24.

26. The nucleic acid according to embodiment 25, comprising a nucleotide sequence having at least 80%, at least 85%, at least 90%, at least 95%, or 100% sequence identity to SEQ ID NO: 5, and/or a nucleotide sequence having at least 80%, at least 85%, at least 90%, at least 95%, or 100% sequence identity to SEQ ID NO:10.

27. The nucleic acid according to embodiment 26, further comprising a nucleotide sequence having at least 80%, at least 85%, at least 90%, at least 95%, or 100% sequence identity to SEQ ID NO: 20, and/or a nucleotide sequence having at least 80%, at least 85%, at least 90%, at least 95%, or 100% sequence identity to SEQ ID NO:31.

28. A vector comprising the nucleic acid according to any one of embodiments 25-27.

29. The vector according to embodiment 28, wherein the vector is selected from the group consisting of lentiviral vectors, retroviral vectors, plasmids, DNA vectors, mRNA vectors, transposon-based vectors and artificial chromosomes.

30. A cell comprising the antigen binding protein according to any one of embodiments 1-24, the nucleic acid according to any one of embodiments 25-27, or the vector according to embodiment 28 or 29.

31. The cell according to embodiment 30, wherein the cell is selected from the group consisting of lymphocytes (e.g. T cells, NK cells), monocytes (e.g. PBMCs) and stem cells.

32. The cell according to embodiment 31, wherein the stem cells are a lymphoid progenitor cells or induced pluripotent stem cells (iPSCs).

33. The cell according to embodiment 31, wherein the cell is a T cell.

34. The cell according to embodiment 33, wherein the T cell does not express an endogenous TCR.

35. A method of preparing the cell of any one of embodiments 30-34, comprising a step of transducing or transfecting a cell with the vector of embodiment 28 or 29.

36. The method according to embodiment 35, further comprising a step of amplifying and/or activating the cell prior to or after the transduction or transfection.

37. A conjugate comprising the antigen binding protein according to any one of embodiments 1-24 and an active agent conjugated or coupled to the antigen binding protein.

38. The conjugate according to embodiment 37, wherein the active agent is selected from the group consisting of a detectable marker, an immunostimulatory molecule and a therapeutic agent;

preferably, the detectable marker is selected from the group consisting of biotins, streptavidin, enzymes or catalytically active fragments thereof, radionuclides, nanoparticles, paramagnetic metal ions, nucleic acid probes, contrast agents, and fluorescent, phosphorescent or chemiluminescent molecules;

preferably, the immunostimulatory molecule is selected from the group consisting of cytokines, chemokines, platelet factors and complement initiators;

preferably, the therapeutic agent is selected from the group consisting of immunomodulators, radioactive compounds, enzymes, chemotherapeutic agents and toxins.

39. A composition comprising the antigen binding protein according to any one of embodiments 1-24, the nucleic acid according to any one of embodiments 25-27, the vector according to embodiment 28 or 29, or the cell according to any one of embodiments 30-34;

preferably, the composition further comprises a pharmaceutically acceptable carrier or excipient.

40. The composition according to embodiment 39, wherein the composition further comprises a second therapeutic agent, preferably the second therapeutic agent is selected from the group consisting of antibodies, chemotherapeutic agents and small molecule drugs.

41. A method of treating or preventing an HPV positive disease in a subject comprising administering to the subject an effective amount of the antigen binding protein according to any one of embodiments 1-24.

42. A method of treating or preventing an HPV positive disease in a subject comprising administering to the subject an effective amount of the cell according to any one of embodiments 30-34.

43. The method according to embodiment 42, wherein the cell is autologous or allogenic for the subject.

44. The method according to embodiment 42, comprising steps of:

(i) isolating a sample containing cells from the subject;

(ii) transducing or transfecting the cells with the vector of embodiment 28 or 29; and

(iii) administering the cells obtained in step (ii) to the subject.

45. The method according to embodiment 44, further comprising a step of knocking out an endogenous TCR in the cells after step (i) and prior to step (ii).

46. The method according to any one of embodiments 41-45, wherein the HPV positive disease is selected from the group consisting of an HPV infection, an HPV premalignancy, and an HPV cancer.

47. The method according to embodiment 46, wherein the cancer is selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

48. The method according to any one of embodiments 41-47, wherein the method further comprises administering a second therapeutic agent, preferably the second therapeutic agent is selected from the group consisting of antibodies, chemotherapeutic agents and small molecule drugs.

49. The method according to any one of embodiments 41-48, wherein the subject has an HLA-A*02 allele, such as HLA-A*02:01, HLA-A*02:03, HLA-A*02:05, HLA-A*02:06, HLA-A*02:07, HLA-A*02:10, or HLA-A*02:11 allele.

50. A method for detecting an HPV positive disease in a subject, wherein the method comprises:

(i) contacting a sample obtained from the subject with the antigen binding protein according to any one of embodiments 1-24, the cell according to any one of embodiments 30-34, or the conjugate according to embodiment 37 or 38; and

(ii) detecting the presence of an HPV antigen in the sample, wherein the presence of the HPV antigen is indicative of the HPV positive disease.

51. The method according to embodiment 50, wherein the HPV positive disease is selected from the group consisting of an HPV infection, an HPV premalignancy and an HPV cancer.

52. The method according to embodiment 51, wherein the cancer is selected from the group consisting of cervical cancer, head and neck cancer, oropharyngeal cancer, esophageal adenocarcinoma, anal cancer, anal canal cancer, rectal cancer, vaginal cancer, vulvar cancer, and penile cancer.

53. A kit comprising the antigen binding protein according to any one of embodiments 1-24or the conjugate according to embodiment 37 or 38, wherein the kit is used for detecting the presence of a positive epitope in a sample to be tested, wherein the epitope is an epitope comprising YMLDLQPET (SEQ ID NO:11).

The present application makes reference to various published patents, published patent applications, journal articles and other publications, all of which are incorporated herein by reference. If there is a conflict between any of the incorporated references and the present specification, the specification prevails.

While the present disclosure has been described with reference to particular embodiments thereof, various changes can be made and equivalents can be substituted without departing from the true spirit and scope of the present disclosure. In addition, many modifications may be made to make particular instances, materials, compositions, methods, steps of methods suitable for the purpose, spirit and scope of the present disclosure. All such modifications are intended to be within the scope of the claims.

1. (canceled)

2. (canceled)

3. An antigen binding protein comprising a T cell receptor (TCR) alpha chain variable region and a TCR beta chain variable region, wherein the TCR alpha chain variable region comprises a CDR3 having the amino acid sequence: AVISAGTALI (SEQ ID NO: 3), or a functional variant thereof formed by insertion, deletion or substitution of one or more amino acids; and/or wherein the TCR beta chain variable region comprises a CDR3 having the amino acid sequence: ASSLGWRGGLYTEAF (SEQ ID NO:8), or a functional variant thereof formed by insertion, deletion or substitution of one or more amino acids,
wherein the antigen binding protein binds to an epitope comprising the amino acid sequence of YMLDLQPET (SEQ ID NO:11) or a complex of the epitope with an MHC molecule.

4. The antigen binding protein according to claim 1, wherein the MHC molecule is HLA-A*02 type, such as HLA-A*02:01 type, HLA-A*02:03 type, HLA-A*02:05 type, HLA-A*02:06 type, HLA-A*02:07 type, HLA-A*02:10 type, or HLA-A*02:11 type.

5. (canceled)

6. The antigen binding protein according to claim 1, wherein the TCR alpha chain variable region comprises CDR1, CDR2 and CDR3 having the amino acid sequences as shown in SEQ ID NO:1, SEQ ID NO:2 and SEQ ID NO:3, respectively, or a functional variant thereof formed by insertion, deletion or substitution of one or more amino acids; and/or the TCR beta chain variable region comprises beta chain CDR1, CDR2 and CDR3 having the amino acid sequences as shown in SEQ ID NO:6, SEQ ID NO:7 and SEQ ID NO: 8, respectively, or a functional variant thereof formed by insertion, deletion or substitution of one or more amino acids.

7. An antigen binding protein comprising a T cell receptor (TCR) alpha chain variable region and a TCR beta chain variable region, wherein the TCR alpha chain variable region comprises CDR1, CDR2, and CDR3 having the amino acid sequences as shown in SEQ ID NO: 1, SEQ ID NO:2, and SEQ ID NO:3, respectively; and/or the TCR beta chain variable region comprises beta chain CDR1, CDR2, and CDR3 having the amino acid sequences as shown in SEQ ID NO:6, SEQ ID NO:7 and SEQ ID NO:8, respectively.8. The antigen binding protein according to any one of claims 1-7, wherein the TCR alpha chain variable region comprises an amino acid sequence having at least 80%, at least 85%, at least 90%, at least 95% or 100% sequence identity to SEQ ID NO:4, and/or the TCR beta chain variable region comprises an amino acid sequence having at least 80%, at least 85%, at least 90%, at least 95% or 100% sequence identity to SEQ ID NO:9.

8. The antigen binding protein according to claim 1, wherein the TCR alpha chain variable region comprises an amino acid sequence having at least 80%, at least 85%, at least 90%, at least 95% or 100% sequence identity to SEQ ID NO:4, and/or the TCR beta chain variable region comprises an amino acid sequence having at least 80%, at least 85%, at least 90%, at least 95% or 100% sequence identity to SEQ ID NO:9.

9. (canceled)

10. The antigen binding protein according to claim 1, wherein the TCR alpha chain variable region is comprised on a first polypeptide and the TCR beta chain variable region is comprised on a different second polypeptide, or the TCR alpha chain variable region and the TCR beta chain variable region are comprised on a single polypeptide.

11. (canceled)

12. The antigen binding protein according to claim 1, wherein the antigen binding protein is soluble or membrane-bound.

13. The antigen binding protein according to claim 1, wherein the antigen binding protein is selected from a TCR or antigen-binding fragment thereof, a chimeric antigen receptor (CAR), a Fc polypeptide, or an antigen-binding fragment thereof.

14. The antigen binding protein according to claim 8, wherein the antigen binding protein further comprises a TCR constant region or a fragment thereof.

15. The antigen binding protein according to claim 9, wherein the TCR constant region is a murine constant region or a human constant region.

16. The antigen binding protein according to claim 9, wherein the TCR constant region comprises a TCR alpha chain constant region and/or a TCR beta chain constant region; and
the TCR alpha chain constant region and/or the TCR beta chain constant region comprises at least one cysteine mutation relative to a wild-type sequence to form a disulfide bond between the TCR alpha chain and the TCR beta chain.

17. The antigen binding protein according to claim 11, wherein the TCR alpha chain constant region comprises an amino acid sequence having at least 80%, at least 85%, at least 90%, at least 95%, or 100% sequence identity to any one of SEQ ID NOs: 14-19, and/or the TCR beta chain constant region comprises an amino acid sequence having at least 80%, at least 85%, at least 90%, at least 95%, or 100% sequence identity to any one of SEQ ID NOs: 21-30.

18. (canceled)

19. (canceled)

20. (canceled)

21. The antigen binding protein according to claim 9, wherein the antigen binding protein comprises a TCR alpha chain comprising an amino acid sequence having at least 80%, at least 85%, at least 90%, at least 95% or 100% sequence identity to any one selected from SEQ ID NOs: 32-37; and/or a TCR beta chain comprising an amino acid sequence having at least 80%, at least 85%, at least 90%, at least 95% or 100% sequence identity to any one selected from SEQ ID NOs: 38-47.

22. (canceled)

23. The antigen binding protein according to claim 1, wherein the antigen binding protein further comprises one or more antigen-binding region(s) that bind(s) to other antigens or epitopes.

24. (canceled)

25. A nucleic acid encoding the antigen binding protein according to claim 1.

26. (canceled)

27. (canceled)

28. A vector comprising the nucleic acid according to claim 15.

29. (canceled)

30. A cell comprising the antigen binding protein according to claim 1, a nucleic acid encoding the antigen binding protein, or a vector comprising the nucleic acid.

31. (canceled)

32. (canceled)

33. (canceled)

34. (canceled)

35. A method of preparing the cell comprising the antigen binding protein according to claim 1, a nucleic acid encoding the antigen binding protein, or a vector comprising the nucleic acid, comprising transducing or transfecting a cell with the vector.

36. (canceled)

37. A conjugate comprising the antigen binding protein according to claim 1 and an active agent conjugated or coupled to the antigen binding protein.

38. (canceled)

39. A composition comprising the antigen binding protein according to claim 1, a nucleic acid encoding the antigen binding protein, a vector comprising the nucleic acid, or a cell comprising the antigen binding protein, the nucleic acid, or the vector.

40. (canceled)

41. A method of treating or preventing an HPV positive disease in a subject comprising administering to the subject an effective amount of the antigen binding protein according to claim 1 or an effective amount of a cell comprising the antigen binding protein, a nucleic acid encoding the antigen binding protein, or a vector comprising the nucleic acid.

42. (canceled)

43. (canceled)

44. (canceled)

45. (canceled)

46. The method according to claim 21, wherein the HPV positive disease is selected from the group consisting of an HPV infection, an HPV premalignancy, and an HPV cancers and the HPV cancer is selected from the group consisting of cervical, cancer head and neck cancer, oropharyngeal cancer, esophageal adenocarcinoma, anal cancer, anal canal cancer, rectal cancer, vaginal cancer, vulvar cancer, and penile cancer.

47. (canceled)

48. (canceled)

49. (canceled)

50. A method for detecting an HPV positive disease in a subject, wherein the method comprises:
contacting a sample obtained from the subject with the antigen binding protein according to claim 1, a cell comprising the antigen binding protein a nucleic acid encoding the antigen binding protein, or a vector comprising the nucleic acid, or a conjugate comprising the antigen binding protein and active agent conjugated or coupled to the antigen binding protein; and
detecting the presence of an HPV antigen in the sample, wherein the presence of the HPV antigen is indicative of the HPV positive disease.

51. (canceled)

52. (canceled)

53. A kit comprising the antigen binding protein according to claim 1 or a conjugate comprising the antigen binding protein and an active agent conjugated or coupled to the antigen binding protein, wherein the kit is used for detecting the presence of a positive epitope in a sample to be tested, wherein the epitope is an epitope comprising YMLDLQPET (SEQ ID NO:11).
